(19) United States
(12) Patent Application Publication
(10) Pub. No.: US 2025/0066508 A1
(43) Pub. Date: Feb. 27, 2025

(54) ANTI-MASP-2 ANTIBODY, PREPARATION METHOD THEREFOR, AND APPLICATION THEREOF

(71) Applicant: SHENYANG SUNSHINE PHARMACEUTICAL CO., LTD., Shenyang City, Liaoning Province (CN)

(72) Inventors: Zhenping ZHU, Shenyang (CN); Chen ZHOU, Shenyang (CN); Haomin HUANG, Shenyang (CN); Jie ZHAO, Shenyang (CN); Liangfeng JIANG, Shenyang (CN); Rui XING, Shenyang (CN)

(21) Appl. No.: 18/722,252

(22) PCT Filed: Dec. 21, 2022
(86) PCT No.: PCT/CN2022/140719

§ 371 Date: Jun. 20, 2024

(30) Foreign Application Priority Data
Dec. 21, 2021 (CN) 202111571729.0

(51) Int. Cl. C07K 16/40; C12N 15/63; G01N 33/577

(52) U.S. Cl. CPC C07K 16/40; C12N 15/63; G01N 33/577; C07K 2317/33; C07K 2317/55; C07K 2317/565; C07K 2317/92; C07K 2319/21

(57) ABSTRACT

The present invention provides an antibody combined with human MASP-2, a preparation method therefor, and an application thereof. The monoclonal antibody of the present invention specifically binds to a MASP-2 antigen, and has high affinity, high specificity and high biological activity, thus having good clinical application prospects.

### TECHNICAL FIELD

The present invention relates to the field of antibody medicaments, specifically, to an anti-MASP-2 antibody, as well as preparation method and use thereof.

### BACKGROUND ART

The lectin pathway is primarily activated by tissue damage or microbial infection. In recent years, more and more studies have shown that the complement-activated MBL pathway plays an important role in a variety of diseases. For example, in patients with IgAN (IgA nephropathy), MBL deposited in glomerular mesangial cells binds to IgA1 and activates MASPs zymogen, thereby activating the MBL pathway. Narsoplimab is a fully human IgG4 monoclonal antibody targeting MASP-2. Currently, Narsoplimab is in Phase III clinical development and is being developed for IgAN and atypical hemolytic uremic syndrome (aHUS). However, the current market still lacks monoclonal antibodies targeting MASP-2 with high affinity, high specificity, and high biological activity.

Therefore, there is a need in this field to develop a monoclonal antibody targeting MASP-2 with high affinity, high specificity, and high biological activity.

### SUMMARY OF THE INVENTION

The purpose of the present invention is to provide an anti-MASP-2 monoclonal antibody with high affinity, high specificity and high biological activity, as well as preparation method and use thereof.

In a first aspect of the present invention, an anti-human MASP-2 antibody or antigen-binding fragment thereof is provided, the antibody or antigen-binding fragment thereof comprising a heavy chain variable region and a light chain variable region, wherein
the heavy chain variable region comprises three heavy chain complementarity determining regions (CDRs):
HCDR1 set forth in SEQ ID NO. 21,
HCDR2 set forth in SEQ ID NO. 22,
HCDR3 set forth in SEQ ID NO. 23; and
the light chain variable region comprises three light chain complementarity determining regions (CDRs):
LCDR1 set forth in SEQ ID NO. 18,
LCDR2 set forth in SEQ ID NO. 19,
LCDR3 set forth in SEQ ID NO. 20;
wherein, any one of the amino acid sequences of the antibody or antigen-binding fragment thereof also comprises a derivative sequence that optionally has been added, deleted, modified and/or substituted by at least one amino acid, and is capable of retaining MASP-2 binding affinity.

In another preferred embodiment, the antigen-binding fragment comprises Fab fragment, F(ab′)₂ fragment, and Fv fragment.

In another preferred embodiment, the amino acid sequence of any of the above-mentioned CDRs comprises a derivative CDR sequence that has been added, deleted, modified and/or substituted by 1, 2, or 3 amino acids, and the derivative antibody composed of VH and VL containing the derivative CDR sequence is capable of retaining binding affinity to MASP-2.

In another preferred embodiment, the number of added, deleted, modified and/or substituted amino acids is 1-5 (such as 1-3, preferably 1-2, more preferably 1).

In another preferred embodiment, the antibody comprises a heavy chain and a light chain, the heavy chain of the antibody comprising the three heavy chain complementarity determining regions (CDRs) and a heavy chain frame region for connecting the heavy chain complementarity determining regions (CDRs); and the light chain of the antibody comprising the three light chain complementarity determining regions (CDRs) and a light chain frame region for connecting the light chain complementarity determining region (CDRs).

In another preferred embodiment, the antibody further comprises a heavy chain constant region and/or a light chain constant region.

In another preferred embodiment, the heavy chain constant region is of human, and/or the light chain constant region is of human.

In another preferred embodiment, the heavy chain variable region of the antibody further comprises a human frame region, and/or the light chain variable region of the antibody further comprises a human frame region.

In another preferred embodiment, the heavy chain variable region of the antibody further comprises a murine frame region, and/or the light chain variable region of the antibody further comprises a murine frame region.

In another preferred embodiment, the heavy chain variable region has the amino acid sequence set forth in SEQ ID NO. 12.

In another preferred embodiment, the heavy chain constant region is of human or murine.

In another preferred embodiment, the heavy chain constant region is a human antibody heavy chain IgG1 or IgG4 constant region.

In another preferred embodiment, the sequence of the heavy chain constant region is set forth in SEQ ID NO. 13.

In another preferred embodiment, the light chain variable region has the amino acid sequence set forth in SEQ ID NO. 11.

In another preferred embodiment, the light chain constant region is of human or murine.

In another preferred embodiment, the light chain constant region is a human antibody light chain kappa or lambda constant region.

In another preferred embodiment, the sequence of the light chain constant region is set forth in SEQ ID NO. 14 or 15.

In another preferred embodiment, the antibody is selected from: animal antibodies, chimeric antibodies, humanized antibodies, fully human antibodies, or combinations thereof.

In another preferred embodiment, the antibody is a partially or fully humanized or fully human monoclonal antibody.

In another preferred embodiment, the antibody is a fully human antibody.

In another preferred embodiment, the antibody is a diabody or a single-chain antibody.

In another preferred embodiment, the antibody is a full-length antibody protein or an antigen-binding fragment.

In another preferred embodiment, the antibody is a monospecific antibody, a bispecific antibody, or a multispecific antibody.

In another preferred embodiment, the antibody is in the form of a drug conjugate.

In another preferred embodiment, the antibody has one or more properties selected from:
(a) it specifically binds to human, mouse, rat or rhesus macaque MASP-2;
(b) the KD value (M) of affinity to human MASP-2 is 1.0E-8 to 1E-10;
(c) it inhibits MASP-2-mediated activation of the downstream complement pathway;
(d) it binds to an epitope in the CCP1/2 domain of MASP-2.

In another preferred embodiment, the amino acid sequence of the heavy chain variable region (VH) is set forth in SEQ ID NO. 12, and the amino acid sequence of the light chain variable region (VL) is set forth in SEQ ID NO. 11.

In another preferred embodiment, the amino acid sequence of the heavy chain variable region has at least 80%, 85%, 90%, 91%, 92%, 93%, 94%, 95%, 96%, 97%, 98% or 99% sequence homology or sequence identity with the amino acid sequence set forth in SEQ ID NO. 12.

In another preferred embodiment, the amino acid sequence of the light chain variable region has at least 80%, 85%, 90%, 91%, 92%, 93%, 94%, 95%, 96%, 97%, 98% or 99% sequence homology or sequence identity with the amino acid sequence set forth in SEQ ID NO. 11.

In a second aspect of the present invention, a recombinant protein is provided, the recombinant protein comprising:
(i) the antibody or antigen-binding fragment thereof according to the first aspect of the present invention; and
(ii) optionally, a tag sequence to assist expression and/or purification.

In another preferred embodiment, the tag sequence comprises 6×His tag.

In another preferred embodiment, the recombinant protein (or polypeptide) comprises a fusion protein.

In another preferred embodiment, the recombinant protein is a monomer, dimer, or multimer.

In another preferred embodiment, the recombinant protein further comprises an additional fusion element (or fusion polypeptide fragment) fused to element (i).

In a third aspect of the present invention, a polynucleotide is provided, the polynucleotide encoding a polypeptide selected from:
(1) the antibody or antigen-binding fragment thereof according to the first aspect of the present invention; or
(2) the recombinant protein according to the second aspect of the present invention.

In a fourth aspect of the present invention, a vector is provided, the vector containing the polynucleotide according to the third aspect of the present invention.

In another preferred embodiment, the vector comprises: bacterial plasmid, phage, yeast plasmid, plant cell virus, mammalian cell virus such as adenovirus, retrovirus, or other vectors.

In a fifth aspect of the present invention, a genetically engineered host cell is provided, the host cell containing the vector according to the fourth aspect of the present invention or having the polynucleotide according to the third aspect of the present invention integrated into the genome.

In a sixth aspect of the present invention, an antibody conjugate is provided, the antibody conjugate containing:
(a) an antibody moiety, such as the antibody or antigen-binding fragment thereof according to the first aspect of the present invention, or combinations thereof; and
(b) a conjugating moiety conjugated to the antibody moiety, the conjugating moiety being selected from: detectable markers, drugs, toxins, cytokines, radionuclides, enzymes, or combinations thereof.

In another preferred embodiment, the conjugate is selected from: fluorescent or luminescent markers, radioactive markers, MRI (magnetic resonance imaging) or CT (computerized X-ray tomography) contrast agents, or enzymes capable of producing detectable products, radionuclides, biotoxins, cytokines (such as IL-2, etc.), antibodies, antibody Fc fragments, antibody scFv fragments, gold nanoparticles/nanorods, virus particles, liposomes, nanomagnetic particles, prodrug-activating enzymes (e.g., DT-diaphorase (DTD) or biphenyl hydrolase-like protein (BPHL)), chemotherapeutic agents (e.g., cisplatin), or any form of nanoparticles, etc.

In another preferred embodiment, the antibody moiety and the conjugating moiety are conjugated through chemical bonds or linkers.

In a seventh aspect of the present invention, a pharmaceutical composition is provided, the pharmaceutical composition containing:
(i) an active ingredient selected from: the antibody or antigen-binding fragment thereof according to the first aspect of the present invention, the recombinant protein according to the second aspect of the present invention, the antibody conjugate according to the sixth aspect of the present invention, or combinations thereof; and
(ii) a pharmaceutically acceptable carrier.

In another preferred embodiment, the pharmaceutical composition further comprises: (iii) other active ingredients for treating MASP-2 related diseases, such as Narsoplimab. In another preferred embodiment, the pharmaceutical composition is a liquid preparation.

In another preferred embodiment, the pharmaceutical composition is an injection.

In another preferred embodiment, the pharmaceutical composition is for treating MASP-2 related diseases.

In an eighth aspect of the present invention, a method for in vitro detection of MASP-2 protein in a sample is provided, the method comprising the steps:
(1) contacting the sample in vitro with the antibody or antigen-binding fragment thereof according to the first aspect of the present invention or the antibody conjugate according to the sixth aspect of the present invention;
(2) detecting whether an antigen-antibody complex is formed, wherein the formation of the complex indicates the presence of MASP-2 protein in the sample.

In a ninth aspect of the present invention, a use of an active ingredient is provided, the active ingredient being selected from: the antibody or antigen-binding fragment thereof according to the first aspect of the present invention, the recombinant protein according to the second aspect of the present invention, the antibody conjugate according to the sixth aspect of the present invention, or the pharmaceutical composition according to the seventh aspect of the present invention, or combinations thereof, the active ingredient being used for:
(a) the preparation of medicaments or preparations for preventing and/or treating MASP-2 related diseases;
(b) inhibiting MASP-2-dependent complement activation; and/or
(c) the preparation of detection reagents or kits.

In a tenth aspect of the present invention, a method for preventing and/or treating MASP-2 related diseases is provided, the method comprising: administering to a subject in need the antibody or antigen-binding fragment thereof according to the first aspect of the present invention, the recombinant protein according to the second aspect of the present invention, the antibody conjugate according to the sixth aspect of the present invention, or the pharmaceutical composition according to the seventh aspect of the present invention, or combinations thereof.

In another preferred embodiment, the MASP-2 related diseases comprise fibrosis or inflammation.

In another preferred embodiment, the MASP-2 related diseases comprise hematological diseases, vascular diseases, renal diseases or renal injuries, ophthalmic diseases, musculoskeletal diseases, gastrointestinal diseases, pulmonary diseases, skin diseases, neurological diseases or injuries, genitourinary diseases, diseases resulting from organ or tissue transplantation surgery, diabetes and diabetic diseases, diseases resulting from chemotherapy and/or radiotherapy treatment, malignancies and endocrine diseases.

In another preferred embodiment, the MASP-2 related diseases are selected from: sepsis, hemorrhagic shock, hemolytic anemia, coagulopathy (such as disseminated intravascular coagulation), cryoglobulinemia, paroxysmal nocturnal hemoglobinuria (PNH); ischemia-reperfusion injury, thrombotic microangiopathy TMA (including hemolytic uremic syndrome (HUS), atypical hemolytic uremic syndrome (aHUS) and thrombotic thrombocytopeniarpura (TTP)), hematopoietic stem cell transplantation-associated thrombotic microangiopathy (HSCT-TMA), catastrophic antiphospholipid syndrome (CAPS), atherosclerosis, myocardial infarction, vasculitis; glomerulonephritis (e.g., Ig A nephropathy), lupus nephritis, membranous nephropathy (MN); age-related macular degeneration (AMD), choroidal neovascularization (CNV), glaucoma, uveitis, retinal vein occlusion; ulcerative colitis, Crohn's disease, pancreatitis, diverticulitis, irritable bowel syndrome; acute respiratory distress syndrome (ARDS), transfusion-related acute lung injury (TRALI), chronic obstructive pulmonary disease (COPD), asthma, diffuse alveolar hemorrhage; stroke, multiple sclerosis (MS), amyotrophic lateral sclerosis (ALS), graft-versus-host disease (GVHD), or combinations thereof.

In another preferred embodiment, the MASP-2 related diseases are selected from: IgA nephropathy, atypical hemolytic uremic syndrome (aHUS), hematopoietic stem cell transplantation-associated thrombotic microangiopathy (HSCT-TMA).

In another preferred embodiment, the aHUS is selected from factor H-independent atypical hemolytic uremic syndrome (aHUS) or atypical hemolytic uremic syndrome (aHUS) secondary to infection.

In another preferred embodiment, the preventing and/or treating comprises reducing the risk of developing the disease, reducing the possibility of clinical symptoms related to the disease, reducing the severity of the disease, and inhibiting the progression of the disease.

In another preferred embodiment, the preventing and/or treating comprises reducing the possibility of at least one of the clinical symptoms (anemia, thrombocytopenia, renal insufficiency and elevated creatinine) associated with atypical hemolytic uremic syndrome (aHUS).

In an eleventh aspect of the present invention, a use of the antibody or antigen-binding fragment thereof according to the first aspect of the present invention in the preparation of a medicament for inhibiting MASP-2-dependent complement activation is provided.

It should be understood that within the scope of the present invention, the above-mentioned technical features of the present invention and the technical features specifically described below (such as in examples) can be combined with each other to form new or preferred technical solutions. Due to space limitations, they will not be described one by one here.

### BRIEF DESCRIPTION OF THE DRAWINGS

FIG. 1 shows a schematic diagram of the molecular structure of MASP.

FIG. 2 shows a schematic diagram of the MASP-mediated complement activation cascade.

FIG. 3 shows the binding effect of anti-MASP-2 antibodies to MASP2-CCP1/2-SP-RKSA.

FIG. 4 shows the binding effect of anti-MASP-2 antibodies to MASP2-CCP1/2-SP-RQ.

FIG. 5 shows the binding effect of anti-MASP-2 antibodies to MASP2-CCP1/2.

FIG. 6 shows the binding effect of anti-MASP-2 antibodies to MASP2-SP-RQSA.

FIG. 7 shows the evaluation-1 of the functional activity of anti-MASP-2 monoclonal antibodies.

FIG. 8 shows the evaluation-2 of the functional activity of anti-MASP-2 monoclonal antibodies.

FIG. 9 shows the ELISA determination of the binding ability of the preferred antibody 169-IgG4 and Narsoplimab to mouse MASP2-SP-RQSA recombinant protein.

FIG. 10 shows the ELISA determination of the binding ability of the preferred antibody 169-IgG4 and Narsoplimab to rat MASP2-SP-RQSA recombinant protein.

FIG. 11 shows the ELISA determination of the binding ability of the preferred antibody 169-IgG4 and Narsoplimab to rhesus macaque MASP2-SP-RQSA recombinant protein.

FIG. 12 shows the ELISA determination of the binding ability of the preferred antibody 169-IgG4 and Narsoplimab to human MASP1-CCP1/2-SP-RQSA.

FIG. 13 shows the ELISA determination of the binding ability of the preferred antibody 169-IgG4 and Narsoplimab to human MASP3-CCP1/2-SP-RQSA.

### DETAILED DESCRIPTION OF THE INVENTION

After extensive and in-depth research and extensive screening, the present inventors have obtained for the first time a series of monoclonal antibodies targeting MASP-2 with high affinity and high specificity wherein the affinity of the preferred antibody is better than that of Narsoplimab in the prior art. In addition, by engineering the MASP-2 antigen, a stably expressed human MASP-2 antigen recombinant protein was obtained. Specifically, using the unique dual display technology and strand displacement technology of Shuangzhan Biotech, an anti-MASP-2 antibody targeting the CCP1/2 domain of the human MASP-2 antigen recombinant protein was obtained, which can effectively inhibit MASP-2-mediated activation of the downstream complement pathway, and its inhibitory ability is stronger than that of Narsoplimab in the prior art; and it has species cross-reactivity and high specificity. On this basis, the present invention was completed.

### Terminology

Unless otherwise defined, all technical and scientific terms used herein have the same meaning as commonly understood by one of ordinary skill in the art to which this invention belongs. As used herein, the term “about” when used in reference to a specifically recited value means that the value may vary by no more than 1% from the recited value. For example, as used herein, the expression “about 100” comprises 99 and 101 and all values therebetween (e.g., 99.1, 99.2, 99.3, 99.4, etc.).

As used herein, the term “optionally” or “optional” means that the subsequently described event or circumstance may occur but does not necessarily occur. For example, “optionally comprising 1-3 antibody heavy chain variable regions” means that the antibody heavy chain variable regions of a specific sequence may be present but are not required to be present, and may be 1, 2, or 3.

As used herein, “contain”, “have” or “comprise” comprises “comprise”, “consist mainly of”, “consist substantially of”, and “consist of”; “consist mainly of”, “consist substantially of”, and “consist of” belong to the specific concepts of “contain”, “have” or “comprise”.

### MBL-Associated Serine Protease 2 (MASP2)

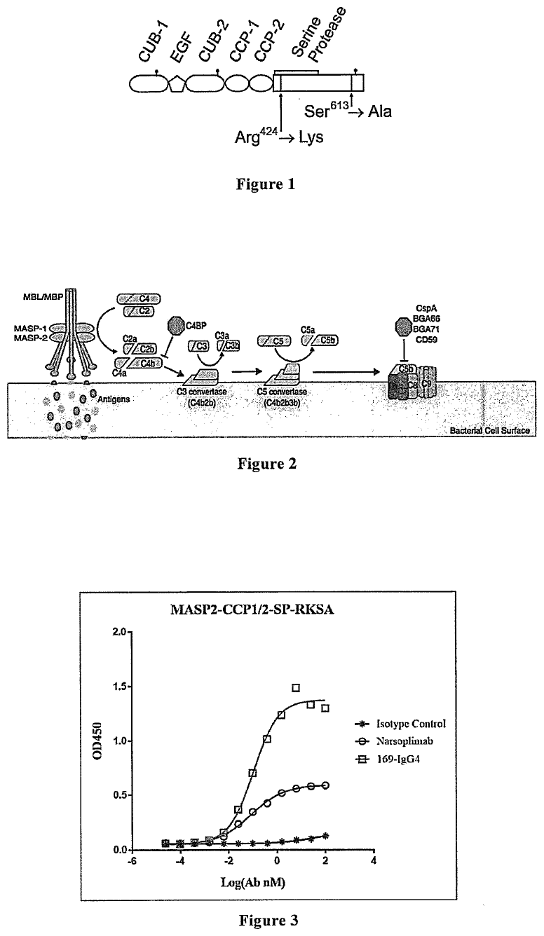

The third pathway of complement activation, the lectin pathway, is an important component of the innate immune system, and mannan-binding lectin (MBL)-associated serine protease 2 (MASP2) is its key protease. Three serine proteases of the MASP family have been found: MASP1, MASP2, and MASP3. All three can form macromolecular complexes (MBL-MASPs) with MBL, among which MASP2 is the main enzyme that activates the MBL pathway. The MASP2 molecule is a single peptide chain, consisting of 6 functional regions from the N-terminus to the C-terminus, which are sequentially: CUB domain (CUB-1), EGF-like domain, the second CUB domain (CUB-2), 2 tandem CCP domains (CCP1 and CCP2), a serine protease (SP) domain. The molecular structure of MASP is shown in FIG. 1 (mutation of Arg at position 424 to Lys and Ser at position 613 to Ala can cause the loss of the protease activity of SP. Reference: Chen C B, Wallis R. Two mechanisms for mannose-binding protein modulation of the activity of its associated serine proteases [J]. Journal of Biological Chemistry, 2004, 279(25): 26058-26065). Studies have shown that the functions of the N-terminus and C-terminus of MASP protein are relatively independent: the three domains at the N-terminus of MASP protein, namely 2 CUB regions and 1 EGF region, are the regions where MASP binds to MBL and can interact with MBL in a Ca²⁺-dependent manner to form MBL-MASPs complex; and the three domains at the C-terminus, namely 2 CCP regions and 1 SP region, are the active centers of the serine protease. Activating complement mainly relies on the SP region to play the role of serine proteases and cleave complements C4 and C2 to produce C3 convertase. In the lectin pathway, MBL/ficolin and MASP1/MASP2 and the like form a C1-like complex. The former recognizes and binds to carbohydrate structure such as mannose, N-acetylglucose, etc. on the surface of pathogenic microorganisms through carbohydrate recognition domain (CRD), and the latter are then activated. The activated MASP2 cleaves C4 and C2 to form C3 convertase (C4bC2a). C3 convertase cleaves C3 into C3a and C3b, thereby forming C5 convertase (C4bC2aC3b). C5 convertase cleaves C5 into C5a and C5b. C5b combines with other complement components to eventually form the membrane attack complex (MAC), leading to cell damage or death. The MASP-mediated complement activation cascade is shown in FIG. 2 (a picture from Stone B L, Brissette C A. Host immune evasion by Lyme and relapsing fever borreliae: findings to lead future studies for Borrelia miyamotoi [J]. Frontiers in immunology, 2017, 8: 12).

In specific embodiments of the present invention, the present anti-MASP-2 antibody binds to portions of full-length human MASP-2 such as CCP1, CCP2 and SP domains; preferably bind to CCP1 and/or CCP2 domain of full-length human MASP-2. In some embodiments, the antibody binds to optimized mutants of the human MASP-2 recombinant protein. Specifically, the present anti-MASP-2 antibody binds to MASP2-CCP1/2-SP-RKSA (SEQ ID NO. 2), MASP2-CCP1/2-SP-RQ (SEQ ID NO. 3), MASP2-CCP1/2 (SEQ ID NO. 4) or MASP2-SP-RQSA (SEQ ID NO. 5). Furthermore, the present anti-MASP-2 antibody has species cross-reactivity and binds to portions of mouse, rat and rhesus macaque MASP-2 or mutants thereof.

### Antibody

In the present invention, the terms “Antibody (Ab)” and “Immunoglobulin G (IgG)” are heterotetrameric proteins with the same structural characteristics, which consist of two identical light chains (L) and two identical heavy chains (H). Each light chain is connected to the heavy chain by a covalent disulfide bond, and the number of disulfide bonds between heavy chains of different immunoglobulin isotypes differs. Each heavy chain and light chain also has regularly spaced intrachain disulfide bonds. Each heavy chain has a variable region (VH) at one end, followed by a constant region. The heavy chain constant region consists of three domains, CH1, CH2, and CH3. Each light chain has a variable region (VL) at one end and a constant region at the other end. The constant region of the light chain includes a domain CL. The light chain constant region pairs with the CH1 domain of the heavy chain constant region, and the variable region of the light chain is paired with the variable region of the heavy chain. Constant regions are not directly involved in the binding of antibodies to antigens, but they exhibit different effector functions, such as participating in antibody-dependent cell-mediated cytotoxicity (ADCC). The heavy chain constant region includes IgG1, IgG2, IgG3, and IgG4 subtypes; and the light chain constant region includes κ (Kappa) or λ (Lambda). The heavy and light chains of the antibody are covalently linked together by the disulfide bond between the CH1 domain of the heavy chain and the CL domain of the light chain. The two heavy chains of the antibody are covalently linked together by the inter-polypeptide disulfide formed between the hinge regions. The present invention includes not only intact antibodies, but also antibody fragments with immunological activity or fusion proteins formed by antibodies and other sequences. Therefore, the present invention also includes fragments, derivatives and analogs of the antibody.

“Monoclonal antibody” as used herein refers to an antibody obtained from a population that is essentially homogeneous, that is, the individual antibodies contained in the population are identical, except for a few naturally occurring mutations that may be present. Monoclonal antibodies target a single antigenic site with high specificity. Furthermore, unlike conventional polyclonal antibody preparations, which typically have different antibodies directed against different determinants, each monoclonal antibody is directed against a single determinant on the antigen. In addition to their specificity, the benefit of monoclonal antibodies is that they are synthesized by hybridoma culture and are not contaminated by other immunoglobulins. The modifier “monoclonal” indicates the nature of the antibody as having been obtained from a substantially homogeneous population of antibodies and should not be construed as requiring any special method to produce the antibody. The monoclonal antibodies can be developed by a variety of approaches and technologies, including hybridoma technology, phage display technology, single lymphocyte gene cloning technology, etc.

The “antigen-binding fragment” of the present invention refers to an antibody fragment capable of specifically binding to human MASP-2. Examples of antigen-binding fragments of the present invention include Fab fragments, F(ab′)₂ fragments, Fv fragments, and the like. Fab fragments are fragments produced by digesting antibodies with papain. F(ab′)₂ fragments are fragments produced by digesting antibodies with pepsin. Fv fragments consist of dimers in which the heavy chain variable region and the light chain variable region of an antibody are tightly non-covalently associated.

In the present invention, the terms “Fab” and “Fc” mean that papain can cleave an antibody into two identical Fab segments and one Fc segment. The Fab segment consists of the VH and CH1 domains of the heavy chain and the VL and CL domains of the light chain of the antibody. The Fc segment is a fragment crystallizable (Fc), which consists of the CH2 and CH3 domains of the antibody. The Fc segment has no antigen-binding activity and is the site where the antibody interacts with effector molecules or cells.

In the present invention, the term “scFv” refers to a single chain antibody fragment (scFv), which consists of an antibody heavy chain variable region and a light chain variable region that are usually connected by a short peptide linker of 15 to 25 amino acids.

In the present invention, the term “variable” means that certain parts of the variable regions of the antibody differ in sequence, which contribute to the binding and specificity of various specific antibodies to their specific antigens. However, variability is not evenly distributed throughout the antibody variable region. It is concentrated in three segments called complementarity determining regions (CDRs) or hypervariable regions in the heavy chain variable regions and light chain variable regions. The more conserved part of the variable region is called the frame region (FR). The variable regions of natural heavy chains and light chains each contain four FR regions, which are roughly in a B-sheet configuration and are connected by three CDRs forming a connecting loop. In some cases, a partial β-sheet structure can be formed. The CDRs in each chain are held closely together by the FR regions and together with the CDRs of the other chain form the antigen-binding site of the antibody (see Kabat et al., NIH Publ. No. 91-3242, Volume I, pp. 647-669 (1991)).

As used herein, the term “frame region” (FR) refers to the amino acid sequences inserted between the CDRs, i.e., those portions of the light chain and heavy chain variable regions of an immunoglobulin that are relatively conserved among different immunoglobulins within a single species. The light chain and heavy chain of immunoglobulins each have four FRs, which are called FR1-L, FR2-L, FR3-L, FR4-L and FR1-H, FR2-H, FR3-H, and FR4-H respectively. Accordingly, the light chain variable domain may thus be referred to as (FR1-L)-(CDR1-L)-(FR2-L)-(CDR2-L)-(FR3-L)-(CDR3-L)-(FR4-L), and the heavy chain variable domain may thus be represented as (FR1-H)-(CDR1-H)-(FR2-H)-(CDR2-H)-(FR3-H)-(CDR3-H)-(FR4-H). Preferably, the FR of the present invention is a human antibody FR or a derivative thereof. The derivative of the human antibody FR is basically the same as the naturally occurring human antibody FR, that is, the sequence identity reaches 85%, 90%, 95%, 96%, 97%, 98% or 99%. Knowing the amino acid sequence of the CDR, those skilled in the art can easily determine the frame regions FR1-L, FR2-L, FR3-L, FR4-L and/or FR1-H, FR2-H, FR3-H, and FR4-H.

As used herein, the term “human frame region” is a frame region that is substantially identical (about 85% or more, specifically 90%, 95%, 97%, 99%, or 100%) to the frame region of a naturally occurring human antibody.

In the present invention, the terms “anti”, “binding” and “specifically binding” refer to the non-random binding reaction between two molecules, such as the reaction between an antibody and the antigen it targets. Typically, the antibody binds the antigen with an equilibrium dissociation constant (KD) of less than about 10⁻⁷ M, such as less than about 10⁻⁸ M, 10⁻⁹ M, 10⁻¹⁰ M, 10⁻¹¹ M, or less. As used herein, the term “KD” refers to the equilibrium dissociation constant of a specific antibody-antigen interaction, which is used to describe the binding affinity between an antibody and an antigen. The smaller the equilibrium dissociation constant, the tighter the antibody-antigen binding is, and the higher the affinity between the antibody and the antigen is. For example, surface plasmon resonance (SPR) is used to determine the binding affinity of an antibody to an antigen in a BIACORE instrument or ELISA is used to determine the relative binding affinity of an antibody to an antigen.

In the present invention, the term “epitope” refers to a polypeptide determinant that specifically binds to an antibody. An epitope of the present invention is a region of an antigen that is bound by an antibody.

In the present invention, antibodies include murine, chimeric, humanized or fully human antibodies prepared using techniques well known to those skilled in the art.

In the present invention, antibodies may be monospecific, bispecific, trispecific, or more multispecific.

In the present invention, the present antibody also includes conservative variants thereof, which refer to polypeptides in which at most 10, preferably at most 8, more preferably at most 5, most preferably at most 3 amino acids are substituted by amino acids with similar properties as compared with the amino acid sequence of the present antibody. Most preferably, these conservative variant polypeptides are produced according to amino acid substitutions in Table A.

TABLE A

| Initial | Representative | Preferable |
|---|---|---|
| residue | substitution | substitution |
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

### Anti-MASP-2 Antibodies

In the present invention, the antibody is an anti-MASP-2 antibody. The present invention provides an antibody against MASP-2 with high specificity and high affinity, which comprises a heavy chain and a light chain, the heavy chain containing the heavy chain variable region (VH) amino acid sequence, and the light chain containing the light chain variable region (VL) amino acid sequence.

Preferably, the heavy chain variable region (VH) comprises the following three complementarity determining regions (CDRs):
HCDR1 set forth in SEQ ID NO. 21,
HCDR2 set forth in SEQ ID NO. 22,
HCDR3 set forth in SEQ ID NO. 23; and
the light chain variable region comprises the following three complementarity determining regions (CDRs):
LCDR1 set forth in SEQ ID NO. 18,
LCDR2 set forth in SEQ ID NO. 19,
LCDR3 set forth in SEQ ID NO. 20;
wherein, any one of the above amino acid sequences also comprises a derivative sequence that optionally has been added, deleted, modified and/or substituted by at least one amino acid, and is capable of retaining MASP-2 binding affinity.

Wherein, any one of the above amino acid sequences also comprises a derivative sequence that optionally has been added, deleted, modified and/or substituted by at least one amino acid, and is capable of retaining MASP-2 binding affinity.

In another preferred embodiment, the sequence formed by adding, deleting, modifying and/or substituting at least one amino acid is preferably an amino acid sequence that has a homology or sequence identity of at least 80%, preferably at least 85%, and more preferably at least 90%, most preferably at least 95%.

Methods for determining sequence homology or identity known to those of ordinary skill in the art include, but are not limited to: Computational Molecular Biology, edited by Lesk, A. M., Oxford University Press, New York, 1988; Biocomputing: Informatics and Genome Projects, edited by Smith, D. W., Academic Press, New York, 1993; Computer Analysis of Sequence Data, Part I, edited by Griffin, A. M. and Griffin, H. G., Humana Press, New Jersey, 1994; Sequence Analysis in Molecular Biology, von Heinje, G., Academic Press, 1987 and Sequence Analysis Primer, edited by Gribskov, M. and Devereux, J., M Stockton Press, New York, 1991 and Carillo, H. and Lipman, D., SIAM J. Applied Math., 48:1073 (1988). The preferred method of determining identity is to obtain the greatest match between the sequences tested. Methods for determining identity are compiled in publicly available computer programs. Preferred computer program methods for determining identity between two sequences include, but are not limited to, the GCG package (Devereux, J. et al., 1984), BLASTP, BLASTN, and FASTA (Altschul, S, F. et al., 1990). The BLASTX program is publicly available from NCBI and other sources (BLAST Manual, Altschul, S. et al., NCBI NLM NIH Bethesda, Md. 20894; Altschul, S. et al., 1990). The well-known Smith Waterman algorithm can also be used to determine identity.

Preferably, the antibody described herein is one or more of a full-length antibody protein, an antigen-antibody binding domain protein fragment, a bispecific antibody, a multispecific antibody, a single chain antibody fragment (scFv), a single domain antibody (sdAb) and a single-domain antibody, as well as monoclonal antibodies or polyclonal antibodies prepared from the above antibodies. The monoclonal antibodies can be developed through a variety of approaches and technologies, including hybridoma technology, phage display technology, single lymphocyte gene cloning technology, etc. The mainstream method is to prepare monoclonal antibodies from wild-type or transgenic mice through hybridoma technology.

The full-length antibody protein is a conventional full-length antibody protein in the art, which comprises a heavy chain variable region, a light chain variable region, a heavy chain constant region and a light chain constant region. The heavy chain variable region and light chain variable region of the protein together with the human heavy chain constant region and the human light chain constant region constitute a fully human full-length antibody protein. Preferably, the full-length antibody protein is IgG1, IgG2, IgG3 or IgG4.

The antibody in the present invention (anti-MASP-2 antibody) can be a full-length protein (such as IgG1, IgG2a, IgG2b or IgG2c), or a protein fragment containing an antigen-antibody binding domain (such as Fab, F(ab′), sdAb, ScFv fragment).

The antibody in the present invention (anti-MASP-2 antibody) can be a wild-type protein, or a mutant protein that has achieved a specific effect through specific mutations, for example, using mutations to eliminate the effector function of the antibody.

The present antibody can be a diabody or a single-chain antibody, and can be selected from animal antibodies, chimeric antibodies, and humanized antibodies, more preferably humanized antibodies, human-animal chimeric antibodies, and more preferably fully humanized antibodies.

The antigen-binding fragment of the present antibody can be a single-chain antibody and/or an antibody fragment, such as: Fab, Fab′, (Fab′)₂ or other antibody derivatives known in the field, as well as any one or more of IgA, IgD, IgE, IgG and IgM antibodies or other subtypes of antibodies.

Among them, the animal is preferably a mammal, such as a murine.

The present antibody may be chimeric antibodies, humanized antibodies, CDR-grafted and/or modified antibodies targeting MASP-2 (e.g., human MASP-2).

In the above content of the present invention, the number of added, deleted, modified and/or substituted amino acids is preferably no more than 40%, more preferably no more than 35%, more preferably 1-33%, more preferably 5-30%, more preferably 10-25%, and more preferably 15-20% of the total number of amino acids in the initial amino acid sequence.

In the above content of the present invention, more preferably, the number of added, deleted, modified and/or substituted amino acids can be 1-7, more preferably 1-5, more preferably 1-3, more preferably 1-2.

In another preferred embodiment, the heavy chain variable region of the antibody has the amino acid sequence set forth in SEQ ID NO. 12.

In another preferred embodiment, the light chain variable region of the antibody has the amino acid sequence set forth in SEQ ID NO. 11.

In another preferred embodiment, the heavy chain variable region (VH) of the antibody targeting MASP-2 has the amino acid sequence set forth in SEQ ID NO. 12, and/or the light chain variable region (VL) has the amino acid sequence set forth in SEQ ID NO. 11.

In another preferred embodiment, the antibody targeting MASP-2 is 169-IgG4.

### Recombinant Protein

The present invention also provides a recombinant protein, which comprises the antibody or antigen-binding fragment thereof according to the first aspect of the present invention, such as comprises one or more of the heavy chain CDR1 (HCDR1), heavy chain CDR2 (HCDR2) and heavy chain CDR3 (HCDR3) of the MASP-2 antibody, and/or one or more of the light chain CDR1 (LCDR1), light chain CDR2 (LCDR2), and light chain CDR3 (LCDR3) of the MASP-2 antibody.

In another preferred embodiment, the recombinant protein further comprises an additional fusion element (or fusion polypeptide fragment) fused to the antibody or antigen-binding fragment thereof.

Wherein, the preparation method of the recombinant protein is a conventional preparation method in this field. The preparation method is preferably: isolating from the expression transformants that recombinantly express the protein or obtaining by artificially synthesizing protein sequences. The preferred method of isolating from the expression transformants that recombinantly expressing the protein is as follows: cloning the nucleic acid molecule encoding the protein and carrying point mutations into a recombinant vector; transforming the obtained recombinant vector into the transformant to obtain recombinantly expressing transformants; culturing the obtained recombinantly expressing transformants; and obtaining the recombinant protein by isolation and purification.

### Encoding Nucleic Acids and Expression Vectors

The invention also provides polynucleotide molecules encoding the above-mentioned antibodies or fragments or fusion proteins thereof. The polynucleotides of the present invention may be in DNA form or RNA form. DNA form includes cDNA, genomic DNA, or artificially synthesized DNA. DNA can be single-stranded or double-stranded. DNA can be a coding strand or a non-coding strand.

The sequence of the DNA molecule of the present antibody or fragment thereof can be obtained using conventional techniques, such as PCR amplification or genome library screening. In addition, the coding sequences of the light and heavy chains can also be fused together to form single-chain antibodies.

Once the relevant sequence is obtained, recombination can be used to obtain the relevant sequence in large quantities. This is usually done by cloning it into a vector, transforming it into cells, and then isolating the relevant sequence from the propagated host cells by conventional methods.

In addition, artificial synthesis methods can also be used to synthesize relevant sequences, especially when the fragment length is short. Often, fragments with long sequences are obtained by first synthesizing multiple small fragments and then ligating them.

At present, the DNA sequence encoding the present antibody (or fragment thereof, or derivative thereof) can be obtained entirely through chemical synthesis. The DNA sequence can then be introduced into a variety of existing DNA molecules (or vectors) and cells known in the art. In addition, mutations can also be introduced into the protein sequence of the present invention through chemical synthesis.

The present invention also relates to vectors comprising appropriate DNA sequences as described above and appropriate promoter or control sequences. These vectors can be used to transform appropriate host cells to enable expression of the protein.

Wherein, the vector is a conventional expression vector in the art, which means an expression vector which contains appropriate regulatory sequences, such as promoter sequences, terminator sequences, polyadenylation sequences, enhancer sequences, marker genes and/or sequences, and other appropriate sequences. The expression vector can be a virus or a plasmid, such as an appropriate phage or phagemid. For more technical details, please refer to, for example, Sambrook et al., Molecular Cloning: A Laboratory Manual, second edition, Cold Spring Harbor Laboratory Press, 1989. Many known techniques and protocols for nucleic acid manipulation are found in Current Protocols in Molecular Biology, 2nd Edition, Ausubel et al. The expression vector of the present invention is preferably pcDNA3.4, pDR1, pcDNA3.1(+), pcDNA3.1/ZEO(+), pDHFR, pcDNA4, pDHFF, pGM-CSF or pCHO 1.0.

In the present invention, the term “host cell” refers to various host cells conventional in the art, as long as the vector can stably replicate itself and the polynucleotide molecules carried can be effectively expressed. Among them, the host cells include prokaryotic expression cells and eukaryotic expression cells, and the host cells preferably include: COS, CHO, NS0, sf9, sf21, DH5a, BL21 (DE3), TG1, BL21 (DE3), 293F or 293E cells.

### Preparation of Antibodies

Typically, the transformed host cells are cultured under conditions suitable for expression of the present antibody. Then conventional immunoglobulin purification steps, such as protein A-Sepharose, hydroxyapatite chromatography, gel electrophoresis, dialysis, ion exchange chromatography, hydrophobic chromatography, molecular sieve chromatography or affinity chromatography and other techniques in the art which are conventional separation and purification means well known to those skilled in the art are used to purify and obtain the present antibody.

The obtained monoclonal antibody can be identified by conventional means. For example, the binding specificity of a monoclonal antibody can be determined using immunoprecipitation or in vitro binding assays such as radioimmunoassay (RIA) or enzyme-linked immunosorbent assay (ELISA). The binding affinity of a monoclonal antibody can be determined, for example, by the Scatchard analysis of Munson et al., Anal. Biochem., 107:220 (1980).

The present antibody can be expressed within cells and secreted outside cells. If desired, the recombinant protein can be isolated and purified by various separation methods utilizing its physical, chemical and other properties. These methods are well known to those skilled in the art. Examples of these methods include, but are not limited to: conventional renaturation treatment, treatment with protein precipitating agents (salting out method), centrifugation, penetrating and breaking bacteria, sonication, ultracentrifugation, molecular sieve chromatography (gel filtration), adsorption chromatography, ion exchange chromatography, high performance liquid chromatography (HPLC) and various other liquid chromatography techniques and combinations of these methods.

### Pharmaceutical Compositions and Applications

The invention also provides a composition. Preferably, the composition is a pharmaceutical composition, which contains the above-mentioned antibody or active fragment thereof or fusion protein thereof, and a pharmaceutically acceptable carrier. Generally, these substances may be formulated in a nontoxic, inert, and pharmaceutically acceptable aqueous carrier medium, usually at a pH of about 5-8, preferably at a pH of about 6-8, although the pH may vary depending on the nature of the substance formulated and the condition to be treated. The prepared pharmaceutical composition can be administered by conventional routes, including (but not limited to): intravenous injection, intravenous drip, subcutaneous injection, local injection, intramuscular injection, intratumoral injection, intraperitoneal cavity injection (such as intraperitoneal injection), intracranial injection, or intracavity injection. In the present invention, the term “pharmaceutical composition” means that the present anti-MASP-2 antibody can be combined with a pharmaceutically acceptable carrier to form a pharmaceutical preparation composition to exert a more stable therapeutic effect. These preparations can ensure that the anti-MASP-2 antibody disclosed in the present invention maintains the conformational integrity of the amino acid core sequence, while also protecting the multifunctional groups of the protein from degradation (including but not limited to aggregation, deamination, or oxidation). The present pharmaceutical composition contains a safe and effective amount (such as 0.001-99 wt %, preferably 0.01-90 wt %, more preferably 0.1-80 wt %) of the above-mentioned anti-MASP-2 antibody (or conjugate thereof) of the present invention and pharmaceutically acceptable carriers or excipients. Such carriers include, but are not limited to: saline, buffer, glucose, water, glycerol, ethanol, and combinations thereof. The pharmaceutical preparation should match the mode of administration. The present pharmaceutical composition can be prepared in the form of an injection, for example, prepared by conventional methods using physiological saline or an aqueous solution containing glucose and other adjuvants. Pharmaceutical compositions such as injections and solutions should be manufactured under sterile conditions. The active ingredient is administered in a therapeutically effective amount, for example, about 10 μg/kg body weight to about 50 mg/kg body weight per day. In addition, the present anti-MASP-2 antibody can also be used in combination with other therapeutic agents, such as other immune molecule modulators.

When using a pharmaceutical composition, a safe and effective amount of an anti-MASP-2 antibody or immunoconjugate thereof is administered to a mammal, wherein the safe and effective amount is usually at least about 10 μg/kg body weight, and in most cases no more than about 50 mg/kg body weight, preferably the dosage is about 10 μg/kg body weight to about 10 mg/kg body weight. Of course, the specific dosage should also take into account factors such as the route of administration and the health condition of the patient, which are all within the skill of a skilled physician.

### Antibody-Drug Conjugates (ADCs)

The present invention also provides an antibody-drug conjugate (ADC) based on the present antibody.

Typically, the antibody-drug conjugate comprises the antibody and an effector molecule, and the antibody is conjugated, and preferably chemically conjugated, to the effector molecule. Among them, the effector molecule is preferably a drug with therapeutic activity. In addition, the effector molecule may be one or more of toxic proteins, chemotherapeutic drugs, small molecule drugs or radionuclides.

The present antibody and the effector molecule can be conjugated through a conjugating agent. Examples of the conjugating agent may be any one or more of non-selective conjugating agents, conjugating agents utilizing carboxyl groups, peptide chains, and conjugating agents utilizing disulfide bonds. The non-selective conjugating agent refers to a compound that allows the effector molecule and the antibody to form a covalent bond, such as glutaraldehyde, etc. The conjugating agent utilizing carboxyl groups may be any one or more of aconitic anhydride conjugating agents (such as aconitic anhydride) and acyl hydrazone conjugating agents (the conjugating site is an acyl hydrazone).

Certain residues on antibodies (such as Cys or Lys, etc.) are used to connect to a variety of functional groups, including imaging reagents (such as chromophores and fluorescent groups), diagnostic reagents (such as MRI contrast agents and radioisotopes), stabilizers (e.g. glycol polymers) and therapeutic agents. Antibodies can be conjugated to functional agents to form antibody-functional agent conjugates. Functional agents (such as drugs, detection reagents, stabilizers) are conjugated (covalently linked) to the antibody. The functional agent can be linked to the antibody directly or indirectly through a linker.

Antibodies can be conjugated with drugs to form antibody-drug conjugates (ADCs). Typically, ADCs contain a linker between the drug and the antibody. Linkers can be degradable or non-degradable linkers. Degradable linkers are typically susceptible to degradation in the intracellular environment, such as at the target site, allowing the drug to be released from the antibody. Suitable degradable linkers include, for example, enzymatically degradable linkers, including peptidyl-containing linkers that can be degraded by intracellular proteases (e.g., lysosomal or endosomal proteases), or sugar linkers, such as those glucuronide-containing linkers that can be degraded by glucuronidases. Peptidyl linkers may include, for example, dipeptides such as valine-citrulline, phenylalanine-lysine or valine-alanine. Other suitable degradable linkers include, for example, pH-sensitive linkers (e.g., linkers that are hydrolyzed at pH less than 5.5, such as hydrazone linkers) and linkers that are degraded under reducing conditions (e.g., disulfide linkers). Non-degradable linkers typically release the drug under conditions in which the antibody is hydrolyzed by proteases.

Before being linked to the antibody, the linker has an active reactive group that can react with certain amino acid residues, and the linking is achieved through the active reactive group. Thiol-specific reactive groups are preferred and include, for example, maleimides, halogenated amides (e.g., iodinated, brominated, or chlorinated); halogenated esters (e.g., iodinated, brominated, or chlorinated); halogenated methyl ketones (e.g. iodinated, brominated, or chlorinated), benzyl halides (e.g. iodinated, brominated, or chlorinated); vinyl sulfone, pyridyl disulfide; mercury derivatives such as 3,6-bis-(mercurymethyl)dioxane, and the counter ion is acetate, chloride or nitrate; and polymethylene dimethyl sulfide thiosulfonate. Linkers may include, for example, maleimides linked to the antibody via thiosuccinimide.

The drug can be any cytotoxic, cytostatic, or immunosuppressive drug. In embodiments, a linker links the antibody and the drug, and the drug has a functional group that can form a bond with the linker. For example, the drug may have an amino, carboxyl, thiol, hydroxyl, or ketone group that can form a bond with the linker. In the case where the drug is directly linked to the linker, the drug has reactive groups before being linked to the antibody.

Useful drug classes include, for example, antitubulin drugs, DNA minor groove binding agents, DNA replication inhibitors, alkylating agents, antibiotics, folate antagonists, antimetabolites, chemosensitizers, topoisomerase inhibitors, Catharanthus roseus alkaloids, etc. In the present invention, drug-linkers can be used to form ADCs in one simple step. In other embodiments, bifunctional linker compounds can be used to form ADCs in a two- or multi-step process. For example, a cysteine residue reacts with the reactive moiety of the linker in a first step, and in a subsequent step, the functional group on the linker reacts with the drug, forming an ADC.

Typically, functional groups on the linker are selected to facilitate specific reaction with appropriate reactive groups on the drug moiety. As a non-limiting example, azide-based moieties can be used to specifically react with reactive alkynyl groups on the drug moiety. The drug is covalently bound to the linker via a 1,3-dipolar cycloaddition between the azide and alkynyl groups. Other useful functional groups include, for example, ketones and aldehydes (suitable for reaction with hydrazides and alkoxyamines), phosphines (suitable for reaction with azides), isocyanates and isothiocyanates (suitable for reactions with amines and alcohols) and activated esters, such as N-hydroxysuccinimide ester (suitable for reactions with amines and alcohols). These and other linking strategies, such as those described in Bioconjugation Technology, 2nd edition (Elsevier), are well known to those skilled in the art. Those skilled in the art can understand that for the selective reaction between a drug moiety and a linker, when a complementary pair of reactive functional groups is selected, each member of the complementary pair can be used for both the linker and the drug.

The present invention also provides a method for preparing an ADC, which may further include: conjugating the antibody to a drug-linker compound under conditions sufficient to form an antibody-drug conjugate (ADC).

In certain embodiments, methods of the present invention comprise conjugating the antibody to a bifunctional linker compound under conditions sufficient to form an antibody-linker conjugate. In these embodiments, the methods of the present invention further comprise: conjugating the antibody-linker conjugate to the drug moiety under conditions sufficient to covalently link the drug moiety to the antibody through the linker.

In some embodiments, the antibody-drug conjugate (ADC) is represented by the following formula:

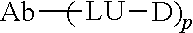

wherein:
Ab is an antibody,
LU is a linker;
D is a drug;
and the subscript p is a value selected from 1 to 8.

### Detecting Uses and Kits

The present antibody or ADC thereof can be used in detection applications, for example in detecting specimen to provide diagnostic information.

In the present invention, the specimens (samples) used include cells, tissue specimens and biopsy specimens. The term “biopsy” as used herein shall include all types of biopsies known to those skilled in the art. Biopsies used in the present invention may thus include, for example, resection specimens of tumors, tissue specimens prepared by endoscopic methods, or puncture or needle biopsy of organs.

Specimens used in the present invention include fixed or preserved cell or tissue specimens.

The invention also provides a kit containing the present antibody (or fragment thereof). In a preferred embodiment of the present invention, the kit further comprises a container, instructions for use, a buffer, etc. In a preferred embodiment, the present antibody can be immobilized on a detection plate.

The present invention also provides a method for detecting MASP-2 protein in a sample (for example, detecting cells that overexpress MASP-2), comprising the following steps: contacting in vitro the above-mentioned antibody with the sample to be tested, and detecting whether the above-mentioned antibody and the sample to be tested combine to form an antigen-antibody complex.

The meaning of overexpression is conventional in the art and refers to the RNA or protein overexpression of MASP-2 protein in the sample to be tested (due to increased transcription, post-transcriptional processing, translation, post-translational processing and protein degradation changes), and local overexpression and increased functional activity (e.g. in the case of increased enzymatic hydrolysis of the substrate) due to altered protein transport patterns (increased nuclear localization).

In the present invention, the method for detecting whether an antigen-antibody complex is formed is a conventional detection method in this field, preferably flow cytometry (FACS) detection.

The present invention provides a composition for detecting MASP-2 protein in a sample, which comprises the above-mentioned antibodies, recombinant proteins, antibody conjugates, immune cells, or combinations thereof as active ingredients. Preferably, it also comprises a compound composed of the functional fragment of the above-mentioned antibody as an active ingredient.

### The Main Advantages of the Present Invention Include

(1) The present anti-MASP-2 antibody has high affinity, high specificity, and high biological activity;
(2) The present anti-MASP-2 antibody can effectively inhibit MASP-2-mediated activation of the downstream complement pathway;
(3) The present anti-MASP-2 antibody has species cross-reactivity.

The present invention will be further described below in conjunction with specific Examples. It should be understood that these examples are only used to illustrate the invention and are not intended to limit the scope of the present invention. Experimental methods without specifying detailed conditions in the following examples usually follow conventional conditions such as those described in Sambrook et al., Molecular Cloning: Laboratory Manual (New York: Cold Spring Harbor Laboratory Press, 1989), or according to suggested conditions from the manufacturer. Unless otherwise stated, percentages and parts are calculated by weight.

The protein expression and purification methods used in the examples are described as follows: the gene of interest was constructed into the expression vector pcDNA3.4, and PEI (Polyethylenimine) was used to transfer the constructed expression vector or a combination of expression vectors into FreeStyle™ 293-F Cells (hereinafter referred to as HEK293F, purchased from Thermo Fisher Scientific) to express antibodies or recombinant proteins. HEK293F cells were cultured in Free Style 293 Expression Medium (purchased from Thermo Fisher Scientific) for 5 days. The cell supernatant was collected, then the antibody was purified with Protein A affinity chromatography, while the recombinant protein was purified by Ni-NTA affinity chromatography.

The enzyme-linked immunosorbent assay (ELISA) method used in the following examples is described as follows: the microplate was coated with the corresponding recombinant protein, and PBST containing 1% bovine serum albumin (PBST is phosphate buffer containing 0.05% Tween-20) was used to block the microplate. The antibody to be tested was serially diluted, then transferred to the above-mentioned microplate coated with recombinant protein, followed by incubation at room temperature for half an hour. After washing the plate, appropriately diluted HRP (Horseradish Peroxidase)-labeled goat anti-human antibody (Fc specific, purchased from Sigma) was added, followed by incubation at room temperature for half an hour. After washing the plate, 100 μl of chromogenic solution with TMB (3,3′,5,5′-Tetramethylbenzidine) was added as substrate to each well, followed by incubation at room temperature for 1 to 5 minutes. 50 μl stop solution (2M H₂SO₄) was added to stop the reaction. OD450 was read by a plate reader (SpectraMax 190). GraphPad Prism7 was used for graphing and data analyzing, and EC₅₀/IC₅₀ was calculated.

The antigen sequences constructed in the following examples are summarized in Table B.

TABLE B
Table of antigen amino acid sequences

| | | SEQ ID |
|---|---|---|
| Name | Sequence | NO. |
| Human | TPLGPKWPEPVFGRLASPGFPGEYANDQERRWTLTAPPGY | 1 |
| MASP-2 | RLRLYFTHFDLELSHLCEYDFVKLSSGAKVLATLCGQEST | |
| full-length | DTERAPGKDTFYSLGSSLDITFRSDYSNEKPFTGFEAFYAA | |
| amino acid | EDIDECQVAPGEAPTCDHHCHNHLGGFYCSCRAGYVLHR | |
| sequence | NKRTCSALCSGQVFTQRSGELSSPEYPRPYPKLSSCTYSISL | |
| | EEGFSVILDFVESFDVETHPETLCPYDFLKIQTDREEHGPFC | |
| | GKTLPHRIETKSNTVTITFVTDESGDHTGWKIHYTSTAQPC | |
| | PYPMAPPNGHVSPVQAKYILKDSFSIFCETGYELLQGHLPL | |
| | KSFTAVCQKDGSWDRPMPACSIVDCGPPDDLPSGRVEYIT | |
| | GPGVTTYKAVIQYSCEETFYTMKVNDGKYVCEADGFWTS | |
| | SKGEKSLPVCEPVCGLSARTTGGRIYGGQKAKPGDFPWQ | |
| | VLILGGTTAAGALLYDNWVLTAAHAVYEQKHDASALDIR | |
| | MGTLKRLSPHYTQAWSEAVFIHEGYTHDAGFDNDIALIKL | |
| | NNKVVINSNITPICLPRKEAESFMRTDDIGTASGWGLTQRG | |
| | FLARNLMYVDIPIVDHQKCTAAYEKPPYPRGSVTANMLCA | |
| | GLESGGKDSCRGDSGGALVFLDSETERWFVGGIVSWGSM | |
| | NCGEAGQYGVYTKVINYIPWIENIISDF | |
| MASP2- | QPCPYPMAPPNGHVSPVQAKYILKDSFSIFCETGYELLQG | 2 |
| CCP1/2- | HLPLKSFTAVCQKDGSWDRPMPACSIVDCGPPDDLPSGRV | |
| SP-RKSA | EYITGPGVTTYKAVIQYSCEETFYTMKVNDGKYVCEADG | |
| amino acid | FWTSSKGEKSLPVCEPVCGLSAKTTGGRIYGGQKAKPGD | |
| sequence | FPWQVLILGGTTAAGALLYDNWVLTAAHAVYEQKHDASA | |
| | LDIRMGTLKRLSPHYTQAWSEAVFIHEGYTHDAGFDNDIA | |
| | LIKLNNKVVINSNITPICLPRKEAESFMRTDDIGTASGWGLT | |
| | QRGFLARNLMYVDIPIVDHQKCTAAYEKPPYPRGSVTAN | |
| | MLCAGLESGGKDACRGDSGGALVFLDSETERWFVGGIVS | |
| | WGSMNCGEAGQYGVYTKVINYIPWIENIISDFGGGGSHH | |
| | HHHH | |
| MASP2- | QPCPYPMAPPNGHVSPVQAKYILKDSFSIFCETGYELLQG | 3 |
| CCP1/2- | HLPLKSFTAVCQKDGSWDRPMPACSIVDCGPPDDLPSGRV | |
| SP-RQ | EYITGPGVTTYKAVIQYSCEETFYTMKVNDGKYVCEADG | |
| amino acid | FWTSSKGEKSLPVCEPVCGLSARTTGGQIYGGQKAKPGDF | |
| sequence | PWQVLILGGTTAAGALLYDNWVLTAAHAVYEQKHDASAL | |
| | DIRMGTLKRLSPHYTQAWSEAVFIHEGYTHDAGFDNDIAL | |
| | IKLNNKVVINSNITPICLPRKEAESFMRTDDIGTASGWGLT | |
| | QRGFLARNLMYVDIPIVDHQKCTAAYEKPPYPRGSVTAN | |
| | MLCAGLESGGKDSCRGDSGGALVELDSETERWFVGGIVS | |
| | WGSMNCGEAGQYGVYTKVINYIPWIENIISDFGGGGSHH | |
| | HHHH | |
| MASP2- | QPCPYPMAPPNGHVSPVQAKYILKDSFSIFCETGYELLQG | 4 |
| CCP1/2 | HLPLKSFTAVCQKDGSWDRPMPACSIVDCGPPDDLPSGRV | |
| amino acid | EYITGPGVTTYKAVIQYSCEETFYTMKVNDGKYVCEADG | |
| sequence | FWTSSKGEKSLPVCEPVCGLSARTTGGGGGGSHHHHHH | |
| MASP2- | QIYGGQKAKPGDFPWQVLILGGTTAAGALLYDNWVLTAA | 5 |
| SP-RQSA | HAVYEQKHDASALDIRMGTLKRLSPHYTQAWSEAVFIHE | |
| amino acid | GYTHDAGFDNDIALIKLNNKVVINSNITPICLPRKEAESFM | |
| sequence | RTDDIGTASGWGLTQRGFLARNLMYVDIPIVDHQKCTAAY | |
| | EKPPYPRGSVTANMLCAGLESGGKDSCRGDAGGALVFLD | |
| | SETERWFVGGIVSWGSMNCGEAGQYGVYTKVINYIPWIE | |
| | NIISDFGGGGSHHHHHH | |
| Mouse | RPCPDPTAPPNGSISPVQAIYVLKDRFSVFCKTGFELLQGS | 6 |
| MASP2- | VPLKSFTAVCQKDGSWDRPMPECSIIDCGPPDDLPNGHVD | |
| CCP1/2- | YITGPEVTTYKAVIQYSCEETFYTMSSNGKYVCEADGFWT | |
| SP-RQSA | SSKGEKLPPVCEPVCGLSTHTIGGQIVGGQPAKPGDFPWQ | |
| amino acid | VLLLGQTTAAAGALIHDNWVLTAAHAVYEKRMAASSLNI | |
| sequence | RMGILKRLSPHYTQAWPEEIFIHEGYTHGAGFDNDIALIKL | |
| | KNKVTINGSIMPVCLPRKEAASLMRTDFTGTVAGWGLTQ | |
| | KGLLARNLMFVDIPIADHQKCTAVYEKLYPGVRVSANML | |
| | CAGLETGGKDACRGDSGGALVFLDNETQRWFVGGIVSW | |
| | GSINCGAADQYGVYTKVINYIPWIENIISNFGGGGSHHHH | |
| | HH | |
| Rat | QPCPDPTAPPNGHISPVQATYVLKDSFSVFCKTGFELLQGS | 7 |
| MASP2- | VPLKSFTAVCQKDGSWDRPIPECSIIDCGPPDDLPNGHVDY | |
| CCP1/2- | ITGPEVTTYKAVIQYSCEETFYTMSSNGKYVCEADGFWTS | |
| SP-RQSA | SKGEKSLPVCKPVCGLSTHTSGGQIIGGQPAKPGDFPWQV | |
| amino acid | LLLGETTAAGALIHDDWVLTAAHAVYGKTEAMSSLDIRM | |
| sequence | GILKRLSLIYTQAWPEAVFIHEGYTHGAGFDNDIALIKLKN | |
| | KVTINRNIMPICLPRKEAASLMKTDFVGTVAGWGLTQKGF | |
| | LARNLMFVDIPIVDHQKCATAYTKQPYPGAKVTVNMLCA | |
| | GLDRGGKDACRGDSGGALVFLDNETQRWFVGGIVSWGSI | |
| | NCGGSEQYGVYTKVTNYIPWIENIINNFGGGGSHHHHHH | |
| Rhesus | QPCPYPMAPPNGHLSPVQAKYILKDSFSIFCEPGYELLQGH | 8 |
| MASP2- | LPLKSFAAVCQKDGSWDQPMPSCSIVDCGPPDDLPSGRVE | |
| CCP1/2- | YITGPEVTTYKAVIQYSCEETFYTMKVNDGKYVCEADGF | |
| SP-RQSA | WTSSKGERSPPVCEPVCGLSARTTGGQIYGGQKAKPGDFP | |
| amino acid | WQVLILGGSTAAGALLYDNWVLTAAHAIYEQKHDASSLDI | |
| sequence | RLGALKRLSPHYTQAWAEAVFIHEGYTHDAGFDNDIALIK | |
| | LNNKVVINSNITPICLPRKEAESFMRTDDIGTASGWGLTQR | |
| | GLLARNLMYVDIPIVDHQKCTAAYEKPPYSGGSVTANML | |
| | CAGLESGGKDACRGDSGGALVFLDNETQRWFVGGIVSW | |
| | GSMNCGEAGQYGVYTKVINYIPWIKNIISNFGGGGSHHHH | |
| | HH | |
| MASP1- | NECPELQPPVHGKIEPSQAKYFFKDQVLVSCDTGYKVLKD | 9 |
| CCP1/2- | NVEMDTFQIECLKDGTWSNKIPTCKIVDCRAPGELEHGLI | |
| SP-RQSA | TFSTRNNLTTYKSEIKYSCQEPYYKMLNNNTGIYTCSAQG | |
| amino acid | VWMNKVLGRSLPTCLPVCGLPKFSRKLMAQIFNGRPAQK | |
| sequence | GTTPWIAMLSHLNGQPFCGGSLLGSSWIVTAAHCLHQSLD | |
| | PEDPTLRDSDLLSPSDFKIILGKHWRLRSDENEQHLGVKH | |
| | TTLHPQYDPNTFENDVALVELLESPVLNAFVMPICLPEGPQ | |
| | QEGAMVIVSGWGKQFLQRFPETLMEIEIPIVDHSTCQKAY | |
| | APLKKKVTRDMICAGEKEGGKDACAGDAGGPMVTLNRE | |
| | RGQWYLVGTVSWGDDCGKKDRYGVYSYIHHNKDWIQR | |
| | VTGVRNGGGGSHHHHHH | |
| MASP3- | NECPELQPPVHGKIEPSQAKYFFKDQVLVSCDTGYKVLKD | 10 |
| CCP1/2- | NVEMDTFQIECLKDGTWSNKIPTCKIVDCRAPGELEHGLI | |
| SP-RQSA | TFSTRNNLTTYKSEIKYSCQEPYYKMLNNNTGIYTCSAQG | |
| amino acid | VWMNKVLGRSLPTCLPECGQPSRSLPSLVKQIIGGRNAEP | |
| sequence | GLFPWQALIVVEDTSRVPNDKWFGSGALLSASWILTAAHV | |
| | LRSQRRDTTVIPVSKEHVTVYLGLHDVRDKSGAVNSSAAR | |
| | VVLHPDFNIQNYNHDIALVQLQEPVPLGPHVMPVCLPRLE | |
| | PEGPAPHMLGLVAGWGISNPNVTVDEIISSGTRTLSDVLQY | |
| | VKLPVVPHAECKTSYESRSGNYSVTENMFCAGYYEGGKD | |
| | TCLGDAGGAFVIFDDLSQRWVVQGLVSWGGPEECGSKQV | |
| | YGVYTKVSNYVDWVWEQMGLPQSVVEPQVERGGGGSH | |
| | HHHHH | |

### Example 1 Preparation of MASP-2 Antigen

The human MASP-2 amino acid sequence is from Uniprot (Entry: 000187). The DNA encoding CCP1, CCP2 and SP domains was synthesized by Shanghai Sangon Bioengineering Co., Ltd., and a coding sequence encoding polyhistidine was added to the end of the gene. Then the recombinant gene was constructed into an expression vector. The resulting gene was named MASP2-CCP1/2-SP. The SP domain has protease activity, is toxic to the expression host, and can cause instability in the recombinant protein itself. Therefore, the Arg at position 424 of MASP-2 was mutated to Lys (R424K), and the Ser at position 613 was mutated to Ala (S613A), which can inactivate the SP domain and enhance the stability of the recombinant protein. The MASP2-CCP1/2-SP carrying these mutations was expressed using the aforementioned method, and then the recombinant protein in the culture supernatant was purified using a Ni-NTA affinity chromatography column. The resulting recombinant protein was named MASP2-CCP1/2-SP-RKSA.

The peptide bond between amino acid residues 428 and 429 of MASP-2 is easily cleaved by protease, and Arg at position 429 of MASP-2 can be mutated to Gln (R429Q) to enhance the stability of the recombinant protein. MASP2-CCP1/2-SP carrying this mutation was expressed and purified using the aforementioned method, and the resulting recombinant protein was named MASP2-CCP1/2-SP-RQ.

The DNA encoding CCP1 and CCP2 was cloned using genetic engineering methods, and a coding sequence encoding polyhistidine was added at the end of the gene. Then the recombinant gene was cloned into an expression vector. The CCP1 and CCP2 domains were expressed and purified using the aforementioned method, and the resulting recombinant protein was named MASP2-CCP1/2.

The DNA encoding the SP domain (containing R429Q and S613A mutations) was cloned using genetic engineering methods. Said mutations can inactivate the SP domain and enhance the stability of the recombinant protein. A coding sequence encoding polyhistidine was added at the end of the gene, and then the recombinant gene was cloned into an expression vector. The SP domain with mutations was expressed and purified using the aforementioned method, and the resulting recombinant protein was named MASP2-SP-RQSA.

### Example 2 Preparation of Anti-MASP-2 Monoclonal Antibody

Unique dual display technology and strand displacement technology of Shuangzhan Biotech (for methods of library construction, please refer to Chinese patent application No. 201910327739.6 or PCT patent application No. PCT/CN2020/085706, for methods of screening functional antibody, please refer to Chinese patent application No. 202110350207.1) was used to construct the heavy chain displacement phage library and the light chain displacement phage library. Screened for anti-MASP-2 antigen-specific Fabs, and a series of anti-MASP-2 antibodies were obtained. Specifically, a total of about 130 ELISA-positive clones were screened, sequenced and analyzed, and 10 clones with better antigen affinity were screened out. Through further screening in terms of biological activity, physical and chemical activity and other aspects, an anti-MASP-2 antibody strain with excellent performance was obtained. The clone number was 169-IgG4 (see Table 1 for sequence), which was used for subsequent analysis and research.

The specific process is as follows:

1. The light chain gene fragment of Narsoplimab and the VH gene fragment library of Shuangzhan Biotech was prepared and inserted into the phage surface Fab display vector, to construct a heavy chain displacement Fab gene library of Narsoplimab; the heavy chain VH gene fragment of Narsoplimab and the LC gene fragment library of Shuangzhan Biotech was prepared and inserted into the phage surface Fab display vector, to construct a light chain Fab displacement gene library of Narsoplimab.

2. The heavy chain displacement gene library and the light chain displacement gene library were introduced into TG1 competent bacteria to construct the corresponding bacterial library.

3. M13KO7 helper phage (NEB, Cat: N0315S) was used to infect the heavy chain displacement bacterial library and the light chain displacement bacterial library, and the heavy chain displacement phage surface Fab display library and the light chain displacement phage surface Fab display library was obtained after packaging and amplification.

4. The biotin-labeled Narsoplimab antigen was mixed with the heavy chain displacement phage surface Fab display library and the light chain displacement phage surface Fab display library respectively. The phage displaying the antigen-specific Fab bound to the biotin-labeled antigen. The binding of magnetic beads labeled with avidin to biotin was used to capture the antigen-specific phage to form a magnetic bead-avidin-biotin-antigen-Fab antibody fragment cross-linked body. Then a pH 2.2 glycine solution was used to elute the phage displaying the MASP-2 antigen-specific Fab from the cross-linked body. Tris buffer at pH 8.0 was used to neutralize to pH 7.0, thereby obtaining a solution of phage displaying MASP-2 antigen-specific heavy chain displacement Fab and light chain displacement Fab.

5. The obtained MASP-2 antigen-specific heavy chain displacement Fab phage and light chain displacement Fab phage was introduced into TG1 bacteria respectively and spread onto the plate to pick colonies. The expression of heavy chain displacement Fab and light chain displacement Fab was amplified and induced. ELISA analysis was conducted for screening. Positive clones expressing MASP-2 antigen-specific heavy chain displacement Fab and light chain displacement Fab was determined by sequencing.

6. The bacterial culture of bacterial clones expressing MASP-2 antigen-specific unique heavy chain displacement Fab determined by sequencing was mixed. The expression vector DNA carried by the bacteria was mini-prepped, and enzyme digested to prepare the screened VH fragment library. The bacterial culture of bacterial clones expressing MASP-2 antigen-specific unique light chain displacement Fab determined by sequencing was mixed. The expression vector DNA carried by the bacteria was mini-prepped, and enzyme digested to prepare the screened full-length light chain library fragments.

7. The screened new heavy chain library fragments and light chain library fragments were inserted into the phage surface Fab display vector to construct a Narsoplimab double displacement Fab gene library.

8. The constructed double displacement Fab gene library was introduced into TG1 competent bacteria to construct the corresponding bacterial library.

9. M13KO7 helper phage was used to infect the double displacement bacterial library. The double displacement phage surface Fab display library was obtained after packaging and amplification. The liquid phase magnetic bead screening method described above was used to screen positive clones expressing MASP-2 antigen-specific double displacement Fab. The double displacement positive clones of unique sequences were determined by sequencing and a series of anti-MASP-2 Fabs were obtained.

10. The screened MASP-2 antigen-specific Fabs were converted into full-length antibodies, and a series of anti-MASP-2 antibodies were obtained after expressing and purifying.

TABLE 1
Amino acid sequences of anti-MASP-2 monoclonal antibodies

| | | | SEQ ID |
|---|---|---|---|
| Antibody No. | | Amino acid sequence | NO. |
| 169-Ig | 169 light | SYELMQPPSMSVSPGQTASITCSGDKLGDIYAY | 11 |
| G4 | chain | WYQQKPGQSPVLVIYQDNKRPSGIPERFSGSN | |
| | variable | SGNTATLTISGTQAMDEADYYCQAWESSTGVF | |
| | region VL | GGGTKLTVL | |
| | 169 heavy | QVQLQESGPGLVKPSETLSLTCTVSGGSISSSS | 12 |
| | chain | YYWGWIRQPPGKGLEWIGSIYYSGSTYYNPSL | |
| | variable | KSRVTISVDTSKNQFSLKLSSVTAADTAVYYCA | |
| | region VH | RDRRGRFDPWGQGTMVTVSS | |
| 169-Ig | LCDR1 | SGDKLGDIYAY | 18 |
| G4 | LCDR2 | QDNKRPS | 19 |
| | LCDR3 | QAWESSTGV | 20 |
| 169-Ig | HCDR1 | SSSYYWG | 21 |
| G4 | HCDR2 | SIYYSGSTYYNPSLKS | 22 |
| | HCDR3 | DRRGRFDP | 23 |
| Amino acid | | ASTKGPSVFPLAPCSRSTSESTAALGCLVKDYF | 13 |
| sequence of human | | PEPVTVSWNSGALTSGVHTFPAVLQSSGLYSLS | |
| IgG4 heavy chain | | SVVTVPSSSLGTKTYTCNVDHKPSNTKVDKRV | |
| constant region | | ESKYGPPCPPCPAPEFLGGPSVFLFPPKPKDTL | |
| | | MISRTPEVTCVVVDVSQEDPEVQFNWYVDGV | |
| | | EVHNAKTKPREEQFNSTYRVVSVLTVLHQDW | |
| | | LNGKEYKCKVSNKGLPSSIEKTISKAKGQPRE | |
| | | PQVYTLPPSQEEMTKNQVSLTCLVKGFYPSDI | |
| | | AVEWESNGQPENNYKTTPPVLDSDGSFFLYSR | |
| | | LTVDKSRWQEGNVFSCSVMHEALHNHYTQKS | |
| | | LSLSLGK | |

| | | |
|---|---|---|
| Amino acid | GQPKAAPSVTLFPPSSEELQANKATLVCLISDF | 14 |
| sequence of human | YPGAVTVAWKADSSPVKAGVETTTPSKQSNNK | |
| Lambda light chain | YAASSYLSLTPEQWKSHRSYSCQVTHEGSTVE | |
| constant region | KTVAPTECS | |
| Amino acid | RTVAAPSVFIFPPSDEQLKSGTASVVCLLNNFY | 15 |
| sequence of human | PREAKVQWKVDNALQSGNSQESVTEQDSKDS | |
| Kappa light chain | TYSLSSTLTLSKADYEKHKVYACEVTHQGLSS | |
| constant region | PVTKSFNRGEC | |
| Amino acid | QVTLKESGPVLVKPTETLTLTCTVSGFSLSRGK | 16 |
| sequence of | MGVSWIRQPPGKALEWLAHIFSSDEKSYRTSL | |
| Narsoplimab heavy | KSRLTISKDTSKNQVVLTMTNMDPVDTATYYC | |
| chain variable | ARIRRGGIDYWGQGTLVTVSS | |
| region | | |
| Amino acid | QPVLTQPPSLSVSPGQTASITCSGEKLGDKYAY | 17 |
| sequence of | WYQQKPGQSPVLVMYQDKQRPSGIPERFSGS | |
| Narsoplimab light | NSGNTATLTISGTQAMDEADYYCQAWDSSTAV | |
| chain variable | FGGGTKLTVL | |
| region | | |

Note:
The bold mark is the variable region determined according to Kabat rules, and the underline mark is the CDR region determined according to Kabat rules. LCDR stands for light chain complementarity determining region, and HCDR stands for heavy chain complementarity determining region.

The DNA of the heavy chain variable region and the complete light chain of the above antibody were synthesized by Shanghai Sangon Bioengineering Co., Ltd. The synthesized heavy chain variable region DNA was connected to the human IgG4 heavy chain constant region DNA to obtain a full-length heavy chain gene. The light chain variable region DNA was connected to the Lambda light chain constant region DNA to obtain the full-length light chain gene. The full-length genes of the above heavy chain and light chain were cloned into expression vectors, and then the antibodies were expressed and purified. The resulting antibodies were named as described in the table above.

Control Narsoplimab is a fully human IgG4 monoclonal antibody targeting MASP-2 developed by Omeros Corporation. The amino acid sequences of its heavy chain and light chain variable region are from “WHO Drug Information, Vol. 33, No. 2, 2019”, respectively identical to SEQ ID NOs 15 and 17 in US20130344073A1. The heavy chain and light chain DNA of Narsoplimab were synthesized by Shanghai Sangon Bioengineering Co., Ltd. The synthesized heavy chain variable region DNA was connected to the human IgG4 heavy chain constant region DNA to obtain the full-length heavy chain gene; the Narsoplimab light chain variable region DNA was connected to the human Lambda light chain constant region DNA to obtain the full-length light chain gene. The full-length genes of the above heavy chain and light chain were cloned into the expression vector pcDNA3.4, and then the antibody was expressed and purified.

### Example 3 Affinity Evaluation of Anti-MASP-2 Monoclonal Antibodies

3.1 Affinity with Antigens MASP2-CCP1/2-SP-RKSA and MASP2-CCP1/2-SP-RQ

Microplates were coated with MASP2-CCP1/2-SP-RKSA and MASP2-CCP1/2-SP-RQ (20 ng/well), and then the binding abilities of 169-IgG4 and Narsoplimab to these two antigens were determined using ELISA.

The ELISA results show (FIG. 3/Table 2 and FIG. 4/Table 3) that the binding effect of 169-IgG4 on these two antigens is not weaker than that of the positive control antibody Narsoplimab. The smaller the EC₅₀ and the larger the Top, the stronger the binding effect is. Among them, Isotype Control is a control antibody that does not bind to the relevant target.

TABLE 2
Binding effect of anti-MASP-2 antibodies
on MASP2-CCP1/2-SP-RKSA

| Antibody | EC50 (nM) | Top |
|---|---|---|
| Narsoplimab | 0.08488 | 0.5904 |
| 169-IgG4 | 0.1059 | 1.378 |

TABLE 3
Binding effect of anti-MASP-2 antibodies
on MASP2-CCP1/2-SP-RQ

| Antibody | EC50 (nM) | Top |
|---|---|---|
| Narsoplimab | 0.102 | 2.015 |
| 169-IgG4 | 0.1355 | 2.264 |

3.2 Affinity with Antigens MASP2-CCP1/2 and MASP2-SP-RQSA

The microplate was coated with MASP2-CCP1/2 and MASP2-SP-RQSA (20 ng/well), and then the binding ability of the preferred antibody (169-IgG4) and Narsoplimab to these two antigens was determined using ELISA.

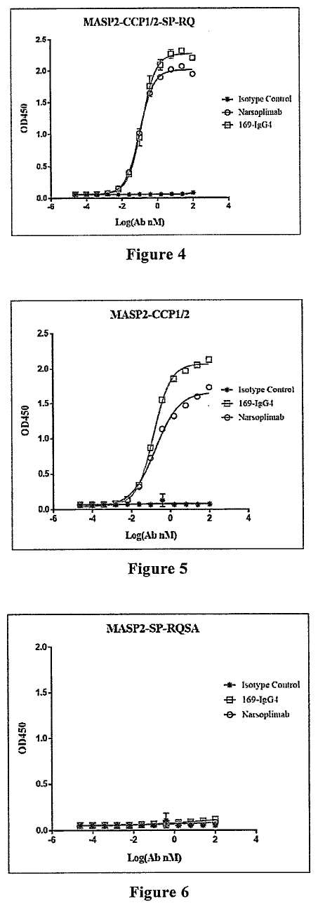

ELISA results show (FIG. 5 and FIG. 6) that 169-IgG4 and Narsoplimab can effectively bind to the CCP1/2 domain of MASP-2. Their EC₅₀ are 0.1464 nM and 0.1772 nM respectively, and Top are 2.069 and 1.664 respectively, which shows that the relative affinity of IgG4-169 is significantly higher than that of Narsoplimab. 169-IgG4 and Narsoplimab do not bind to the SP domain of MASP-2. Among them, Isotype Control is a control antibody that does not bind to the relevant target.

### Example 4 Evaluation of Functional Activity of Anti-MASP-2 Monoclonal Antibodies

After MBL binds to mannan, it can activate the protease activity of MASP-2, and the activated MASP-2 further mediates the activation of the downstream complement pathway. This example evaluates the inhibitory effect of 169-IgG4 on MASP-2-mediated activation of the complement pathway.

The specific implementation method is described as follows: mannan (purchased from Merk, product number: M7504) was dissolved in carbonate buffer (pH 9.5) to prepare a 40 μg/ml or 100 μg/ml mannan solution. Microplates were coated with this solution (50 μL per well). The antibody sample to be tested was diluted with GVB buffer (containing 4 mM barbiturate, 141 mM NaCl, 1 mM MgCl₂, 2 mM CaCl₂) and 0.1% gelatin, pH7.4). Fresh human plasma with a final concentration of 1% was added to the solution, and anti-MASP-2 antibodies to be tested were added and serially diluted. This mixture solution was transferred into a mannan-coated microplate (100 μL per well) and incubated at room temperature for 30 min to activate the complement system. The microplate was transferred to an ice bath to stop the reaction, and was immediately washed three times with PBST. Appropriately diluted anti-C3 antibody (Polyclonal Rabbit Anti-Human C3 Complement, purchased from Agilent, Cat. No.: F020102) was added to the microplate. After incubating at room temperature for 30 minutes; washing 3 times with PBST, appropriately diluted HRP-conjugated goat anti-rabbit secondary antibody (purchased from Merk, Cat. No.: WBKLS0500) was added to the microplate, and incubated at room temperature for 1 hour. After washing three times with PBST, TMB chromogenic solution (100 μl/well) was added to the microplate. After incubating at room temperature for 5˜15 min, 50 μl stop solution (2M H₂SO₄) was added to terminate the reaction. OD450 was read by a plate reader (SpectraMax 190). GraphPad Prism7 was used for graphing and data analyzing and IC₅₀ was calculated.

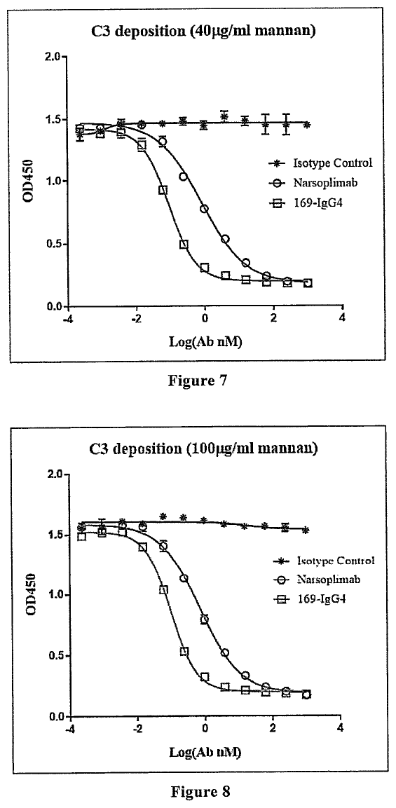

Experimental results (FIGS. 7 and 8) show that Narsoplimab and 169-IgG4 can effectively reduce the deposition of C3 on microplates. At 40 μg/ml mannan, their IC₅₀ were 0.8046 nM and 0.08885 nM respectively; At 100 μg/ml mannan, their IC₅₀ were 0.7298 nM and 0.09962 nM respectively. This indicates that they effectively inhibit MASP-2-mediated activation of the downstream complement pathway, and the inhibitory activity of 169-IgG4 is stronger than that of Narsoplimab.

### Example 5 Species Cross-Reactivity and Specificity of Anti-MASP-2 Monoclonal Antibodies

The MASP-2 amino acid sequences of mice, rats and rhesus macaque are from Uniprot, and the entries are Q91WP0-1, Q9JJS8-1 and F6SW75-1 respectively. The amino acid sequences of human MASP-1 and MASP-3 are also from Uniprot, and the entries are P48740-1 and P48740-2 respectively. The DNA encoding CCP1, CCP2 and SP domains of the above species was synthesized by Shanghai Sangon Bioengineering Co., Ltd., and a coding sequence encoding polyhistidine was added to the end of the gene. Then the recombinant gene was constructed into an expression vector. Mutations similar to the above-mentioned R429Q and S613A were introduced at homologous positions. The mutated SP domain was expressed and purified using the aforementioned method, and the resulting recombinant proteins were named Mouse/Rat/Rhesus MASP2-CCP1/2-SP-RQSA, MASP1-CCP1/2-SP-RQSA and MASP3-CCP1/2-SP-RQSA respectively.

The microplate was coated with the above-mentioned recombinant proteins (Mouse/Rat/Rhesus MASP2-CCP1/2-SP-RQSA, MASP1-CCP1/2-SP-RQSA and MASP3-CCP1/2-SP-RQSA) (20 ng/well), and then ELISA was used to determine the binding ability of the preferred antibody (169-IgG4) and Narsoplimab to these five antigens respectively.

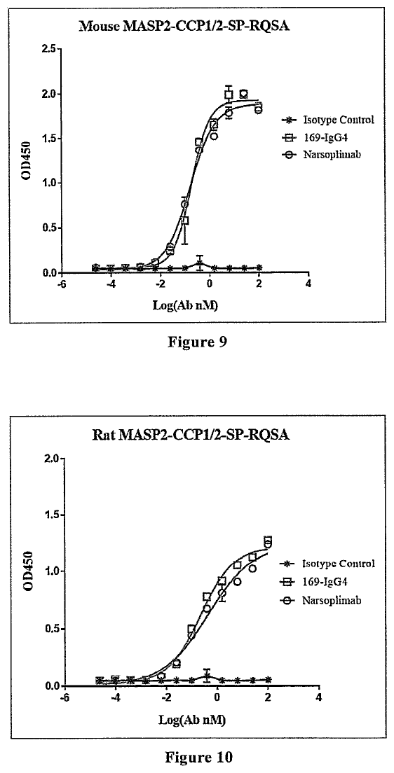

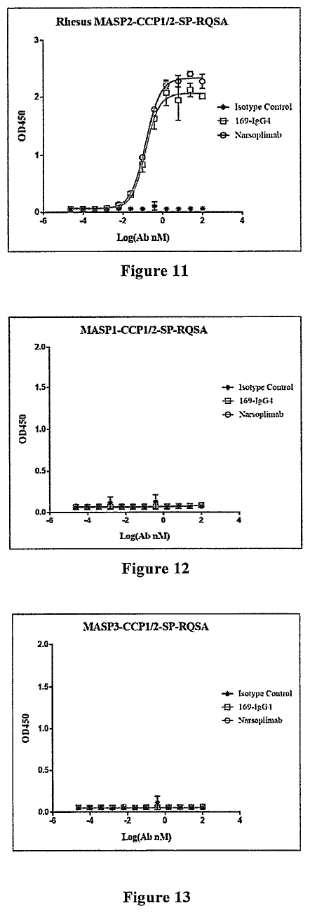

ELISA results show (FIG. 9, FIG. 10 and FIG. 11, Table 4) that both 169-IgG4 and Narsoplimab can effectively bind to mouse, rat and rhesus macaque MASP-2. Among them, Isotype Control is a control antibody that does not bind to the relevant target.

The ELISA results show (FIG. 12 and FIG. 13) that neither 169-IgG4 nor Narsoplimab can bind to human MASP-1 and MASP-3, which indicates that 169-IgG4 has good specificity. Among them, Isotype Control is a control antibody that does not bind to the relevant target.

TABLE 4
Species cross-reactivity of anti-MASP-2 monoclonal antibodies

| | EC50 (nM) | | |
|---|---|---|---|
| Antibody | Mouse | Rat | Rhesus |
| 169-IgG4 | 0.1903 | 0.2314 | 0.136 |
| Narsoplimab | 0.1686 | 0.4552 | 0.1415 |

### Example 6 Biacore Determination of Affinity of the Preferred Anti-MASP-2 Monoclonal Antibodies

Here, the affinity between the above antibodies and MASP2-CCP1/2-SP-RKSA or MASP2-CCP1/2-SP-RQ was detected by Biacore 8K (GE healthcare). On Biacore 8K, a chip conjugated with Protein A/G was used to capture various antibodies, and then the above two recombinations were used as analytes (Analyte) to flow through the chip to obtain the binding-dissociation curve, and regeneration buffer was used to regenerate the chip and proceed to the next cycle; Biacore 8K Evaluation Software was used to analyze the data.

TABLE 5
Affinity of anti-MASP-2 monoclonal
antibodies to MASP2-CCP1/2-SP-RKSA

| | ka | kd | KD |
|---|---|---|---|
| Antibody | (1/Ms) | (1/s) | (M) |
| Narsoplimab | 1.89E+05 | 2.71E−02 | 1.43E−07 |
| 169-IgG4 | 9.92E+04 | 3.82E−03 | 3.86E−08 |

TABLE 6
Affinity of anti-MASP-2 monoclonal
antibodies to MASP2-CCP1/2-SP-RQ

| | ka | kd | KD |
|---|---|---|---|
| Antibody | (1/Ms) | (1/s) | (M) |
| Narsoplimab | 7.30E+05 | 2.06E−02 | 2.83E−08 |
| 169-IgG4 | 7.50E+05 | 3.61E−03 | 4.82E−09 |

Note:
ka: binding constant; kd: dissociation constant; KD: equilibrium dissociation constant; KD = kd/ka.

Experimental results (Table 5 and Table 6) show that the equilibrium dissociation constants (KD) of 169-IgG4 for the above two antigens are the smallest, and the KDs are 3.86E-08 and 4.82E-09 respectively, which indicates the affinity of 169-IgG4 is higher than that of Narsoplimab.

All documents mentioned in this application are incorporated by reference in this application to the same extent as if each individual document was individually incorporated by reference. In addition, it should be understood that after reading the above teaching of the present invention, those skilled in the art can make various changes or modifications to the present invention, and these equivalents also fall within the scope defined by the appended claims of this application.

1. An anti-human MASP-2 antibody or antigen-binding fragment thereof, characterized in that the antibody or antigen-binding fragment thereof comprises a heavy chain variable region and a light chain variable region, wherein
the heavy chain variable region comprises three heavy chain complementarity determining regions (CDRs):
HCDR1 set forth in SEQ ID NO. 21,
HCDR2 set forth in SEQ ID NO. 22,
HCDR3 set forth in SEQ ID NO. 23; and
the light chain variable region comprises three light chain complementarity determining regions (CDRs):
LCDR1 set forth in SEQ ID NO. 18,
LCDR2 set forth in SEQ ID NO. 19,
LCDR3 set forth in SEQ ID NO. 20;
wherein, any one of the amino acid sequences of the antibody or antigen-binding fragment thereof also comprises a derivative sequence that optionally has been added, deleted, modified and/or substituted by at least one amino acid, and is capable of retaining MASP-2 binding affinity.

2. The antibody of claim 1, characterized in that the antibody comprises a heavy chain and a light chain, and the heavy chain of the antibody comprises the three heavy chain complementarity determining regions (CDRs) and a heavy chain frame region for connecting the heavy chain complementarity determining regions (CDRs); and the light chain of the antibody comprises the three light chain complementarity determining regions (CDRs) and a light chain frame region for connecting the light chain complementarity determining regions (CDRs).

3. The antibody of claim 1, characterized in that the amino acid sequence of the heavy chain variable region (VH) is set forth in SEQ ID NO. 12, and the amino acid sequence of the light chain variable region (VL) is set forth in SEQ ID NO. 11.

4. A recombinant protein, characterized in that the recombinant protein comprises:
(i) the antibody or antigen-binding fragment thereof according to claim 1; and
(ii) optionally, a tag sequence to assist expression and/or purification.

5. A polynucleotide, characterized in that the polynucleotide encodes
the antibody or antigen-binding fragment thereof according to claim 1.

6. A vector, characterized in that the vector contains the polynucleotide according to claim 5.

7. A genetically engineered host cell, characterized in that the host cell contains the vector according to claim 6.

8. An antibody conjugate, characterized in that the antibody conjugate contains:
(a) an antibody moiety, such as the antibody or antigen-binding fragment thereof according to claim 1, or combinations thereof; and
(b) a conjugating moiety conjugated to the antibody moiety, the conjugating moiety being selected from: detectable markers, drugs, toxins, cytokines, radionuclides, enzymes, or combinations thereof.

9. A pharmaceutical composition, characterized in that the pharmaceutical composition contains
the antibody or antigen-binding fragment thereof according to claim 1 and
a pharmaceutically acceptable carrier.

10. A method for in vitro detection of MASP-2 protein in a sample, characterized in that the method comprises the steps:
(1) contacting the sample in vitro with the antibody or antigen-binding fragment thereof according to claim 1; and
(2) detecting whether an antigen-antibody complex is formed, wherein the formation of the complex indicates the presence of MASP-2 protein in the sample.

11. A method of inhibiting MASP-2-dependent complement activation, which comprises contacting MASP-2 with the antibody or antigen-binding fragment thereof according to claim 1.

12. A method of treating and/or preventing a MASP-2 related disease, which comprises administering to a patient in need thereof the antibody or antigen-binding fragment thereof according to claim 1.

13. The method of claim 12, wherein the MASP-2 related disease is selected from: hematological diseases, vascular diseases, renal diseases or renal injuries, ophthalmic diseases, musculoskeletal diseases, gastrointestinal diseases, pulmonary diseases, skin diseases, neurological diseases or injuries, genitourinary diseases, diseases resulting from organ or tissue transplantation surgery, diabetes and diabetic diseases, diseases resulting from chemotherapy and/or radiotherapy treatment, malignancies, endocrine diseases, or combination thereof.

14. The method of claim 12, wherein the MASP-2 related disease is selected from: IgA nephropathy, atypical hemolytic uremic syndrome (aHUS), or hematopoietic stem cell transplantation-associated thrombotic microangiopathy (HSCT-TMA).

15. (canceled)


SEQUENCE LISTING (23 sequences)

SEQ ID NO: 1 - protein, 671 aa, synthetic construct
  source 1..671: note = human MASP-2 full length amino acid sequence
TPLGPKWPEPVFGRLASPGFPGEYANDQERRWTLTAPPGYRLRLYFTHFDLELSHLCEYDFVKLSSGAKVLATLCGQESTDTERAPGKDTFYSLGSSLDITFRSDYSNEKPFTGFEAFYAAEDIDECQVAPGEAPTCDHHCHNHLGGFYCSCRAGYVLHRNKRTCSALCSGQVFTQRSGELSSPEYPRPYPKLSSCTYSISLEEGFSVILDFVESFDVETHPETLCPYDFLKIQTDREEHGPFCGKTLPHRIETKSNTVTITFVTDESGDHTGWKIHYTSTAQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 2 - protein, 400 aa, synthetic construct
  source 1..400: note = MASP2-CCP1/2-SP-RKSA amino acid sequence
QPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSAKTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDACRGDSGGALVFLDSETERWFVGGIVSWGSMNCGEAGQYGVYTKVINYIPWIENIISDFGGGGSHHHHHH

SEQ ID NO: 3 - protein, 400 aa, synthetic construct
  source 1..400: note = MASP2-CCP1/2-SP-RQ amino acid sequence
QPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGQIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDFGGGGSHHHHHH

SEQ ID NO: 4 - protein, 157 aa, synthetic construct
  source 1..157: note = MASP2-CCP1/2 amino acid sequence
QPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGGGGGSHHHHHH

SEQ ID NO: 5 - protein, 254 aa, synthetic construct
  source 1..254: note = MASP2-SP-RQSA amino acid sequence
QIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDAGGALVFLDSETERWFVGGIVSWGSMNCGEAGQYGVYTKVINYIPWIENIISDFGGGGSHHHHHH

SEQ ID NO: 6 - protein, 399 aa, synthetic construct
  source 1..399: note = Mouse MASP2-CCP1/2-SP-RQSA amino acid sequence
RPCPDPTAPPNGSISPVQAIYVLKDRFSVFCKTGFELLQGSVPLKSFTAVCQKDGSWDRPMPECSIIDCGPPDDLPNGHVDYITGPEVTTYKAVIQYSCEETFYTMSSNGKYVCEADGFWTSSKGEKLPPVCEPVCGLSTHTIGGQIVGGQPAKPGDFPWQVLLLGQTTAAAGALIHDNWVLTAAHAVYEKRMAASSLNIRMGILKRLSPHYTQAWPEEIFIHEGYTHGAGFDNDIALIKLKNKVTINGSIMPVCLPRKEAASLMRTDFTGTVAGWGLTQKGLLARNLMFVDIPIADHQKCTAVYEKLYPGVRVSANMLCAGLETGGKDACRGDSGGALVFLDNETQRWFVGGIVSWGSINCGAADQYGVYTKVINYIPWIENIISNFGGGGSHHHHHH

SEQ ID NO: 7 - protein, 399 aa, synthetic construct
  source 1..399: note = Rat MASP2-CCP1/2-SP-RQSA amino acid sequence
QPCPDPTAPPNGHISPVQATYVLKDSFSVFCKTGFELLQGSVPLKSFTAVCQKDGSWDRPIPECSIIDCGPPDDLPNGHVDYITGPEVTTYKAVIQYSCEETFYTMSSNGKYVCEADGFWTSSKGEKSLPVCKPVCGLSTHTSGGQIIGGQPAKPGDFPWQVLLLGETTAAGALIHDDWVLTAAHAVYGKTEAMSSLDIRMGILKRLSLIYTQAWPEAVFIHEGYTHGAGFDNDIALIKLKNKVTINRNIMPICLPRKEAASLMKTDFVGTVAGWGLTQKGFLARNLMFVDIPIVDHQKCATAYTKQPYPGAKVTVNMLCAGLDRGGKDACRGDSGGALVFLDNETQRWFVGGIVSWGSINCGGSEQYGVYTKVTNYIPWIENIINNFGGGGSHHHHHH

SEQ ID NO: 8 - protein, 400 aa, synthetic construct
  source 1..400: note = Rhesus MASP2-CCP1/2-SP-RQSA amino acid sequence
QPCPYPMAPPNGHLSPVQAKYILKDSFSIFCEPGYELLQGHLPLKSFAAVCQKDGSWDQPMPSCSIVDCGPPDDLPSGRVEYITGPEVTTYKAVIQYSCEETFYTMKVNDGKYVCEADGFWTSSKGERSPPVCEPVCGLSARTTGGQIYGGQKAKPGDFPWQVLILGGSTAAGALLYDNWVLTAAHAIYEQKHDASSLDIRLGALKRLSPHYTQAWAEAVFIHEGYTHDAGFDNDIALIKLNNKVVINSNITPICLPRKEAESFMRTDDIGTASGWGLTQRGLLARNLMYVDIPIVDHQKCTAAYEKPPYSGGSVTANMLCAGLESGGKDACRGDSGGALVFLDNETQRWFVGGIVSWGSMNCGEAGQYGVYTKVINYIPWIKNIISNFGGGGSHHHHHH

SEQ ID NO: 9 - protein, 412 aa, synthetic construct
  source 1..412: note = MASP1-CCP1/2-SP-RQSA amino acid sequence
NECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPVCGLPKFSRKLMAQIFNGRPAQKGTTPWIAMLSHLNGQPFCGGSLLGSSWIVTAAHCLHQSLDPEDPTLRDSDLLSPSDFKIILGKHWRLRSDENEQHLGVKHTTLHPQYDPNTFENDVALVELLESPVLNAFVMPICLPEGPQQEGAMVIVSGWGKQFLQRFPETLMEIEIPIVDHSTCQKAYAPLKKKVTRDMICAGEKEGGKDACAGDAGGPMVTLNRERGQWYLVGTVSWGDDCGKKDRYGVYSYIHHNKDWIQRVTGVRNGGGGSHHHHHH

SEQ ID NO: 10 - protein, 441 aa, synthetic construct
  source 1..441: note = MASP3-CCP1/2-SP-RQSA amino acid sequence
NECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPECGQPSRSLPSLVKQIIGGRNAEPGLFPWQALIVVEDTSRVPNDKWFGSGALLSASWILTAAHVLRSQRRDTTVIPVSKEHVTVYLGLHDVRDKSGAVNSSAARVVLHPDFNIQNYNHDIALVQLQEPVPLGPHVMPVCLPRLEPEGPAPHMLGLVAGWGISNPNVTVDEIISSGTRTLSDVLQYVKLPVVPHAECKTSYESRSGNYSVTENMFCAGYYEGGKDTCLGDAGGAFVIFDDLSQRWVVQGLVSWGGPEECGSKQVYGVYTKVSNYVDWVWEQMGLPQSVVEPQVERGGGGSHHHHHH

SEQ ID NO: 11 - protein, 106 aa, synthetic construct
  source 1..106: note = 169-IgG4 antibody light chain variable region(VL)
SYELMQPPSMSVSPGQTASITCSGDKLGDIYAYWYQQKPGQSPVLVIYQDNKRPSGIPERFSGSNSGNTATLTISGTQAMDEADYYCQAWESSTGVFGGGTKLTVL

SEQ ID NO: 12 - protein, 118 aa, synthetic construct
  source 1..118: note = 169-IgG4 antibody heavy chain variable region (VH)
QVQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARDRRGRFDPWGQGTMVTVSS

SEQ ID NO: 13 - protein, 327 aa, synthetic construct
  source 1..327: note = human IgG4 heavy chain constant region amino acid sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 106 aa, synthetic construct
  source 1..106: note = Lambda light chain constant region amino acid sequence
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 15 - protein, 107 aa, synthetic construct [sequence omitted]

SEQ ID NO: 16 - protein, 118 aa, synthetic construct
  source 1..118: note = Narsoplimab heavy chain variable region amino acid sequence
QVTLKESGPVLVKPTETLTLTCTVSGFSLSRGKMGVSWIRQPPGKALEWLAHIFSSDEKSYRTSLKSRLTISKDTSKNQVVLTMTNMDPVDTATYYCARIRRGGIDYWGQGTLVTVSS

SEQ ID NO: 17 - protein, 106 aa, synthetic construct
  source 1..106: note = Narsoplimab light chain variable region amino acid sequence
QPVLTQPPSLSVSPGQTASITCSGEKLGDKYAYWYQQKPGQSPVLVMYQDKQRPSGIPERFSGSNSGNTATLTISGTQAMDEADYYCQAWDSSTAVFGGGTKLTVL

SEQ ID NO: 18 - protein, 11 aa, synthetic construct
  source 1..11: note = 169-IgG4 LCDR1
SGDKLGDIYAY

SEQ ID NO: 19 - protein, 7 aa, synthetic construct
  source 1..7: note = 169-IgG4 LCDR2
QDNKRPS

SEQ ID NO: 20 - protein, 9 aa, synthetic construct
  source 1..9: note = 169-IgG4 LCDR3
QAWESSTGV

SEQ ID NO: 21 - protein, 7 aa, synthetic construct
  source 1..7: note = 169-IgG4 HCDR1
SSSYYWG

SEQ ID NO: 22 - protein, 16 aa, synthetic construct
  source 1..16: note = 169-IgG4 HCDR2
SIYYSGSTYYNPSLKS

SEQ ID NO: 23 - protein, 8 aa, synthetic construct
  source 1..8: note = 169-IgG4 HCDR3
DRRGRFDP